(19) United States
(12) Patent Application Publication
(10) Pub. No.: US 2003/0229018 A1
(43) Pub. Date: Dec. 11, 2003

(54) Dimeric TF antagonist

(76) Inventors: Marianne Kjalke, Kobenhavn N (DK); Palle Jakobsen, Vaerlose (DK); Henning Ralf Stennicke, Kokkedal (DK)

Correspondence Address: Reza Green, Esq., Novo Nordisk Pharmaceuticals, Inc., 100 College Road West, Princeton, NJ, 08540, US

(21) Appl. No.: 10/386,898

(22) Filed: Mar. 12, 2003

Related U.S. Application Data
(60) Provisional application No. 60/365,935, filed on Mar. 19, 2002.

(30) Foreign Application Priority Data
Mar. 12, 2002 (DK) PA 2002 00373

(51) Int. Cl. A61K 38/36

(52) U.S. Cl. 514/12

(57) ABSTRACT

Dimer FVII polypeptides, which binds and inhibits two TF molecules simultaneously.

### FIELD OF THE INVENTION

This invention relates to novel compounds which binds to and inhibit the activity of TF. The invention also relates to pharmaceutical compositions comprising the novel compounds as well as their use in treatment of or prophylaxis of thrombotic or coagulopathic related diseases or disorders including vascular diseases and inflammatory responses.

### BACKGROUND OF THE INVENTION

Blood coagulation is a process consisting of a complex interaction of various blood components, or factors, which eventually gives rise to a fibrin clot. Generally, the blood components which participate in what has been referred to as the coagulation “cascade” are proenzymes or zymogens, enzymatically inactive proteins, which are converted to proteolytic enzymes by the action of an activator, itself an activated clotting factor. Coagulation factors that have undergone such a conversion and generally referred to as “active factors”, and are designated by the addition of the letter “a” to the name of the coagulation factor (e.g. FVIIa).

In order to form a fibrin clot and thereby stop a bleeding, activated factor X (FXa) is required. FXa is needed to convert prothrombin to thrombin, which then converts fibrinogen to fibrin as a final stage in forming a fibrin clot. There are two systems, or pathways that promote the activation of factor X (FX). The “intrinsic pathway” refers to those reactions that lead to thrombin formation through utilisation of factors present only in plasma. A series of protease-mediated activations ultimately generates factor IXa, which, in conjunction with factor VIIIa, cleaves FX into FXa. Factor VIIa (FVIIa) and its cofactor tissue factor (TF) in the “extrinsic pathway” of blood coagulation effect an identical proteolysis. While the relative importance of the two coagulation pathways in haemostasis is unclear, FVIIa and TF have in recent years been found to play a pivotal role in the initiation of blood coagulation.

FVIIa is a two-chain, 50 kilodalton (kDa) vitamin-K dependent, plasma serine protease which participates in the complex regulation of in vivo haemostasis. FVIIa is generated from proteolysis of a single peptide bond from its single chain zymogen, Factor VII (FVII), which is present at a concentration of approximately 0.5 μg/ml in plasma. The zymogen is catalytically inactive. The conversion of zymogen FVII into the activated two-chain molecule occurs by cleavage of an internal peptide bond. In human FVII, the cleavage site is at Arg152-Ile153.

In the presence of calcium ions, FVIIa binds with high affinity to exposed TF, an integral membrane protein. TF acts as a cofactor for FVIIa, enhancing the proteolytic activation of its substrates FVII, factor IX and FX.

TF is a 263 amino acid residue glycoprotein composed of a 219 residue extracellular domain, a single transmembrane domain, and a short cytoplasmic domain. TF is a membrane bound protein and does not normally circulate in plasma in an active form.

It is often desirable to selectively block or inhibit the coagulation cascade in a patient, for example during kidney dialysis, or to prevent or treat deep vein thrombosis, sepsis, DIC, atherosclerosis and a host of other medical disorders. Typically, anticoagulants such as heparin, coumarin, derivatives of coumarin, indandione derivatives, thrombin inhibitors, or FXa inhibitors have been used. For example, heparin treatment or extracorporal treatment with citrate ions may be used in dialysis to prevent coagulation during the course of treatment. Heparin is also used in preventing deep vein thrombosis in patients undergoing surgery. Treatment with heparin and other anticoagulants may, however, have undesirable side effects. Available anticoagulants generally act throughout the body, rather than acting specifically at the site of injury, i.e., the site at which the coagulation cascade is active. Heparin, for example, may cause severe bleedings. Furthermore, with a half-life of approximately 80 minutes, heparin is rapidly cleared from the blood, necessitating frequent administrating. Because heparin acts as a cofactor for antithrombin III (ATIII), and ATIII is rapidly depleted in DIC treatment, it is often difficult to maintain the proper heparin dosage, necessitating continuous monitoring of ATIII and heparin levels. Heparin is also ineffective if AT III depletion is extreme. Further, prolonged use of heparin may also increase platelet aggregation and reduce platelet count, and has been implicated in the development of osteoporosis. Indandione derivatives may also have toxic side effects.

Other known anticoagulants comprise thrombin and FXa inhibitors derived from bloodsucking organisms. Antithrombins, hirudin, hirulog and hirugen are recombinant proteins or peptides derived from the leach Hirudo medicinalis, whereas the FXa inhibitor antistatin and the recombinant derivative rTAP are tick-derived proteins. Inhibitors of platelet aggregation, such as monoclonal antibodies or synthetic peptides, which interfere with the platelet receptor GPIIb/IIIa are also effective as anticoagulants.

Bleeding complications are observed as an undesired major disadvantage of anti-thrombin, anti-FXa, as well as anti-platelet reagents. This side effect is strongly decreased or absent with inhibitors of the TF/FVIIa activity. Such anticoagulants comprise the physiological inhibitor TFPI (tissue factor pathway inhibitor) and inactivated FVII (FVIIai), which is FVIIa modified in such a way that it is catalytically inactive and thus not able to catalyse the conversion of FX to FXa, but still able to bind to TF in competition with active endogenous FVIIa.

International patent applications WO 92/15686, WO 94/27631, WO 96/12800, WO 97/47651 relates to FVIIai and the uses thereof. International patent applications WO 90/03390, WO 95/00541, WO 96/18653, and European Patent EP 500800 describes peptides derived from FVIIa having TF/FVIIa antagonist activity. WO 01/21661 relates to bivalent inhibitor of FVII and FXa. WO 02/02764 relates to high molecular weight derivatives of vitamin K-dependent polypeptides.

Hu Z and Garen A (2001) Proc. Natl. Acad. Sci. USA 97; 9221-9225; Hu Z and Garen A (1999) Proc. Natl. Acad. Sci. USA 96; 8161-8166, and WO 0102439 relates to immunoconjugates which comprises the Fc region of a human IgG1 immunoglobulin and a mutant FVII polypeptide, that binds to TF but do not initiate blood clotting.

Furthermore, International patent application WO 98/03632 describes bivalent agonists having affinity for one or more G-coupled receptors, and Burgess, L. E. et al., Proc. Natl. Acad. Sci. USA 96, 8348-8352 (July 1999) describes “Potent selective non-peptidic inhibitors of human lung tryptase”.

There is still a need in the art for improved compositions having anticoagulant activity at relatively low doses and which does not produce undesirable side effects. The present invention provides anticoagulants that act specifically on the TF initiated process at sites of injury at relatively low doses.

### SUMMARY OF THE INVENTION

In a broad aspect, the present invention relates to dimeric TF antagonists. This relates to dimeric FVII polypeptides, which are divalent and bind and inhibit two TF molecules simultaneously, thereby having an avidity effect and possibly increased in vivo half-life.

In a first aspect, the present invention relates to a compound having the formula A-(LM)-D, wherein A and D are FVII polypeptides that bind to TF; and LM is a linker moiety with a molecular weight less than 30,000 daltons; and wherein the compound inhibits TF activity; with the proviso that the compound does have the formula wtFVIIai-(DPTA-dim-FFR-cmk)-wtFVIIai or FVIIai(Qcmk10E32)-(DPTA-dim-FFR-cmk)-FVIIai(Q10E32).

In a second aspect, the present invention relates to a method for reducing TF activity, the method comprising contacting a TF expressing cell with a compound having the for-mula A-(LM)-D, wherein A and D are FVII polypeptides that binds to TF; and LM is a linker moiety with a molecular weight less than 30,000 daltons; and wherein the compound inhibits TF activity; with the proviso that the compound does have the formula wtFVIIai-(DPTA-dim-FFR-cmk)-wtFVIIai or FVIIai(Q10E32)-(DPTA-dim-FFR-cmk)-FVIIai(Q10E32).

In a third aspect, the present invention relates to a pharmaceutical composition comprising an amount of the compound having the formula A-(LM)-D, wherein A and D are FVII polypeptides that binds to TF; and LM is a linker moiety with a molecular weight less than 30,000 daltons; and wherein the compound inhibits TF activity; with the proviso that the compound does have the formula wtFVIIai-(DPTA-dim-FFR-cmk)-wtFVIIai or FVIIai (Q10E32)-(DPTA-dim-FFR-cmk)-FVIIai(Q10E32); and a pharmaceutically accept-able carrier or excipient.

In a further aspect, the present invention relates to a compound for use as a medicament having the formula A-(LM)-D, wherein A and D are FVII polypeptides that binds to TF; and LM is a linker moiety with a molecular weight less than 30,000 daltons; and wherein the compound inhibits TF activity; with the proviso that the compound does have the formula wtFVIIai-(DPTA-dim-FFR-cmk)-wtFVIIai or FVIIai(Q10E32)-(DPTA-dim-FFR-cmk)-FVIIai(Q10E32).

In a further aspect, the present invention relates to the use of a compound having the formula A-(LM)-D, wherein A and D are FVII polypeptides that binds to TF; and LM is a linker moiety with a molecular weight less than 30,000 daltons; and wherein the compound inhibits TF activity; with the proviso that the compound does have the formula wtFVIIai-(DPTA-dim-FFR-cmk)-wtFVIIai or FVIIai(Q10E32)-(DPTA-dim-FFR-cmk)-FVIIai(Q10E32) for the manufacture of a medicament for preventing or treating a TF related diseases or disorders.

In a further aspect, the present invention relates to a method for prevention or treatment of TF related diseases or disorders in a mammal, which method comprises administering to a mammal an effective amount of at least one compound having the formula A-(LM)-D, wherein A and D are FVII polypeptides that binds to TF; and LM is a linker moiety with a molecular weight less than 30,000 daltons; and wherein the compound inhibits TF activity; with the proviso that the compound does have the formula wtFVIIai-(DPTA-dim-FFR-cmk)-wtFVIIai or FVIIai(Q10E32)-(DPTA-dim-FFR-cmk)-FVIIai(Q10E32).

### BRIEF DESCRIPTION OF THE DRAWINGS

FIG. 1 shows a graphic illustration of size exclusion chromatography of rFVIIa reacted with octanedioic acid bis-({1-[1-(1-chloroacetyl-4-guanidino-butylcarbamoyl)-2-phenyl-ethylcarbamoyl]-2-phenyl-ethyl}-amide (a), and a photographic illustration of SDS-PAGE of the fractions (b).

FIG. 2 shows a graphic illustration of the effect of FFR-FVIIa active site dimer and FFR-FVIIa monomer in a competition clotting assay using lipidated recombinant TF (Innovin) as TF source.

FIG. 3 illustrates DNA and amino acid sequences used in the present invention.

### DESCRIPTION OF THE INVENTION

The present invention relates to dimeric FVII polypeptides, which are divalent and binds and inhibits two TF molecules simultaneously, thereby having an avidity effect and possibly increased in vivo half-life. Therefore lower concentration of the TF antagonist can be used as compared to a monovalent FVIIai molecule. In one specific embodiment of the invention, two molecules of FVIIa are linked together with a divalent chloromethyl ketone inhibitor consisting of two chloromethyl ketone moieties linked together.

The inactivated dimeric FVII polypeptide consists of two FVII polypeptides A and D, linked by a suitable linking moiety (LM), said LM being chemically bound to the FVII polypeptides. Compared to inhibitors targeting only one TF site, a dimeric inhibitors provide additional advantages in terms of higher binding affinity and potency due to an avidity effect.

In order to achieve multiple binding, proper design of the TF dimeric antagonist is essential. The functional affinity of such an antagonist is dependent on the overall concentration but particularly on the spatial localisation of the FVII polypeptides A and D, which favours the binding of both motifs of the divalent FVIIai molecule to TF. After binding of one FVII polypeptide to TF, the diffusion of its tethered partner will be constrained within a radius equal to the length of the spacer connecting the two FVII polypeptides. This constraining effect will increase the local concentration for the FVII polypeptide binding to a second TF site.

The terms “TF antagonist” or “TF antagonists”, as used herein is intendend to mean any compound binding directly to TF and inhibiting the conversion of FX to FXa in a FXa generation assay. One example of a TF antagonist is FVIIai.

The terms “TF dimeric antagonist” or “TF dimeric antagonists”, as used herein is intendend to mean any TF antagonist which comprises two binding sites for TF.

A divalent TF inhibitor contains three distinct motifs that can be modulated in order to achieve preferential inhibiting characteristics: the two FVII polypeptides, and the linker moiety (LM) joining the two FVII polypeptides. The LM should not be regarded only as a spacer that connects the two FVII polypeptides. It is possible to utilise the LM for optimising for solubility, bioavailability and for the binding properties of the divalent TF inhibitor in order to obtain the highest affinity.

It is an object of the present invention to provide compounds having pharmacological activity as TF antagonists, and thus to provide methods for inhibiting TF-mediated coagulation activity.

The term “TF-mediated coagulation activity” means coagulation initiated by TF through the formation of the TF/FVIIa complex and its activation of FIX and Factor X to FIXa and FXa, respectively.

The compounds are useful for the prevention or treatment of TF-related diseases or disorders including vascular diseases and inflammatory responses.

ABBREVIATIONS USED THROUGHOUT THE DESCRIPTION INCLUDE:

| | |
|---|---|
| TF | tissue factor |
| FVIIa | activated factor VII |
| FXa | factor Xa, activated factor X |
| FVII | zymogen (single-strand, non-activated) factor VII |
| FX | zymogen (single-strand, non-activated) factor X |
| TF/FVIIa | complex between TF and FVIIa |
| TF/FVIIa/FXa | complex formed by FVIIa, TF and FXa |
| PT | Prothrombin time |
| APTT | Activated partial thromboplastin time |
| SP | serine protease |
| FFR | Phe-Phe-Arg or D-Phe-Phe-Arg |
| D-FFR | D-Phe-Phe-Arg |
| dim-FFR | dimeric form of FFR, two FFRs bound together by linking moiety |
| dim-FFR-FVIIa | dim-FFR bound to factor VIIa, preferably in active site |
| dim-FFR-cmk | dimeric form of FFR-cmk, two FFR-cmk's bound together by link- |
| | ing moiety |
| FFR-cmk, FFR-CMK | Phe-Phe-Arg chloromethyl ketone or D-Phe-Phe-Arg chloromethyl |
| | ketone |
| D-FFR-cmk, D-FFR-CMK | D-Phe-Phe-Arg chloromethyl ketone |
| DPTA-dim-FFR-cmk | diethylenetriaminepentaacetic acid anhydride-dim-FFR-cmk |
| EGR | Glu-Gly-Arg or D-Glu-Gly-Arg |
| D-EGR | D-Glu-Gly-Arg |
| EGR-cmk, EGR-CMK | Glu-Gly-Arg chloromethylketone or D-Glu-Gly-Arg chloromethyl |
| | ketone |
| K_{I} | dissociation constant, inhibition constant of enzyme-inhibitor com- |
| | plex |

It is to be understood that when the designation “D” immediately precedes a letter abreviation for an amino acid as defined above, that amino acid is the non-natural denantiomer.

The term “active site” and the like when used herein with reference to FVIIa refer to the catalytic and zymogen substrate binding site, including the “S₁” site of FVIIa as that term is defined by Schecter, I. and Berger, A., (1967) Biochem. Biophys. Res. Commun. 7:157-162.

A TF/FVIIa mediated or associated process or event, or a process or event associated with TF-mediated coagulation activity, is any event, which requires the presence of TF/FVIIa.

Such processes or events include, but are not limited to, formation of fibrin which leads to thrombus formation; platelet deposition; proliferation of smooth muscle cells (SMCs) in the vessel wall, such as, for example, in intimal hyperplasia or restenosis, which is thought to result from a complex interaction of biological processes including platelet deposition and thrombus formation, release of chemotactic and mitogenic factors, and the migration and proliferation of vascular smooth muscle cells into the intima of an arterial segment; and deleterious events associated with post-ischemic reperfusion, such as, for example, in patients with acute myocardial infarction undergoing coronary thrombolysis.

The no-reflow phenomenon, that is, lack of uniform perfusion to the microvasculature of a previously ischemic tissue has been described for the first time by Krug et al., (Circ. Res. 1966; 19:57-62).

The general mechanism of blood clot formation is reviewed by Ganong, in Review of Medical Physiology, 13^{th} ed., Lange, Los Altos Calif., pp 411-414 (1987). Coagulation requires the confluence of two processes, the production of thrombin which induces platelet aggregation and the formation of fribrin which renders the platelet plug stable. The process comprises several stages each requiring the presence of discrete proenzymes and profactors. The process ends in fibrin crosslinking and thrombus formation. Fibrinogen is converted to fibrin by the action of thrombin. Thrombin, in turn, is formed by the proteolytic cleavage of prothrombin. This proteolysis is effected by FXa which binds to the surface of activated platelets and in the presence of FVa and calcium, cleaves prothrombin. TF/FVIIa is required for the proteolytic activation of FX by the extrinsic pathway of coagulation. Therefore, a process mediated by or associated with TF/FVIIa, or an TF-mediated coagulation activity includes any step in the coagulation cascade from the formation of the TF/FVIIa complex to the formation of a fibrin platelet clot and which initially requires the presence of TF/FVIIa. For example, the TF/FVIIa complex initiates the extrinsic pathway by activation of FX to FXa, FIX to FIXa, and additional FVII to FVIIa. TF/FVIIa mediated or associated process, or TF-mediated coagulation activity can be conveniently measured employing standard assays such as those described in Roy, S., (1991) J. Biol. Chem. 266:4665-4668, and O'Brien, D. et al., (1988) J. Clin. Invest. 82:206-212 for the conversion of FX to FXa in the presence of TF/FVIIa and other necessary reagents.

The term “TF related diseases or disorders” as used herein means any disease or disorder, where TF is involved. This includes, but are not limited to diseases or disorders related to TF-mediated coagulation activity, thrombotic or coagulopathic related diseases or disorders or diseases or disorders such as inflammatory responses and chronic thromboembolic diseases or disorders associated with fibrin formation, including vascular disorders such as deep venous thrombosis, arterial thrombosis, post surgical thrombosis, coronary artery bypass graft (CABG), percutaneous transdermal coronary angioplasty (PTCA), stroke, cancer, tumour metastasis, angiogenesis, ischemia/reperfusion, arthritis including rheumatoid arthritis, thrombolysis, arteriosclerosis and restenosis following angioplasty, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis, and other diseases. The TF related diseases or disorders are not limited to in vivo coagulopatic disorders such as those named above, but includes ex vivo TF/FVIIa related processes such as coagulation that may result from the extracorporeal circulation of blood, including blood removed in-line from a patient in such processes as dialysis procedures, blood filtration, or blood bypass during surgery.

“Treatment” means the administration of an effective amount of a therapeutically active compound of the invention with the purpose of preventing any symptoms or disease state to develop or with the purpose of curing or easing such symptoms or disease states already developed. The term “treatment” is thus meant to include prophylactic treatment.

In a first aspect, the present invention relates to a compound having the formula A-(LM)-D, wherein A and D are FVII polypeptides that binds to TF; and LM is a linker moiety with a molecular weight less than 30,000 daltons; and wherein said compound inhibits TF activity with the proviso that the compound is not a compound having the formula of wtFVIIai-(DPTA-dim-FFR-cmk)-wtFVIIai or FVIIai(Q10E32)-(DPTA-dim-FFR-cmk)-FVIIai(Q10E32).

It is to be understood that moiety A are chemically bound to LM, which is also chemically bound to moiety D. A and D may be identical FVII polypeptides linked together by the LM moiety or A and D may be different FVII polypeptides.

The terms “FVII polypeptide” or “FVII polypeptides” as used herein means without limitation, Factor VII, as well as equivalents of Factor VII. Factor VII equivalents include, without limitation, Factor VII polypeptides that have either been chemically modified relative to human Factor VII and/or contain one or more amino acid sequence alterations relative to human Factor VII (i.e., Factor VII variants), and/or contain truncated amino acid sequences relative to human Factor VII (i.e., Factor VII fragments). Such equivalents may exhibit different properties relative to human Factor VII, including stability, phospholipid binding, altered specific activity, and the like.

The term “Factor VII” is intended to encompass Factor VII polypeptides in their uncleaved (zymogen) form, as well as those that have been proteolytically processed to yield their respective bioactive forms, which may be designated Factor Vila. Typically, Factor VII is cleaved between residues 152 and 153 to yield Factor VIIa. The term “Factor VII” is also intended to encompass, without limitation, polypeptides having the amino acid sequence 1-406 of wild-type human Factor VII (as disclosed in U.S. Pat. No. 4,784,950), as well as wild-type Factor VII derived from other species, such as, e.g., bovine, porcine, canine, murine, and salmon Factor VII. It further encompasses natural allelic variations of Factor VII that may exist and occur from one individual to another. Also, degree and location of glycosylation or other post-translation modifications may vary depending on the chosen host cells and the nature of the host cellular environment.

As used herein, “Factor VII equivalent” encompasses, without limitation, equivalents of Factor VII exhibiting substantially the same or improved biological activity relative to wild-type human Factor VII, as well as equivalents, in which the Factor VIIa biological activity has been substantially modified or reduced relative to the activity of wild-type human Factor VIIa. These polypeptides include, without limitation, Factor VII or Factor VIIa that has been chemically modified and Factor VII variants into which specific amino acid sequence alterations have been introduced that modify or disrupt the bioactivity of the polypeptide.

The term “tissue factor activity” or “TF activity” as used herein means the activity of TF measured in a FXa generation assay.

The term “FXa generation assay” as used herein is intended to mean any assay where activation of FX is measured in a sample comprising TF, FVIIa, FX, calcium and phospholipids. An example of a FXa generation assay is described in assay 2.

In a second aspect, the present invention relates to a method for reducing TF activity, said method comprising contacting a TF expressing cell with a compound having the formula A-(LM)-D, wherein A and D are FVII polypeptides that binds to TF; and LM is a linker moiety with a molecular weight less than 30,000 daltons; and wherein said compound inhibits TF activity with the proviso that the compound is not a compound having the formula of wtFVIIai-(DPTA-dim-FFR-cmk)-wtFVIIai or FVIIai(Q10E32)-(DPTA-dim-FFR-cmk)-FVIIai(Q10E32).

In a third aspect, the present invention relates to a pharmaceutical composition comprising an amount of the compound having the formula A-(LM)-D, wherein A and D are FVII polypeptides that binds to TF; and LM is a linker moiety with a molecular weight less than 30,000 daltons; and wherein said compound inhibits TF activity with the proviso that the compound is not a compound having the formula of wtFVIIai-(DPTA-dim-FFR-cmk)-wtFVIIai or FVIIai(Q10E32)-(DPTA-dim-FFR-cmk)-FVIIai(Q10E32); and a pharmaceutically acceptable carrier or excipient.

In a further aspect, the present invention relates to a compound for use as a medicament having the formula A-(LM)-D, wherein A and D are FVII polypeptides that binds to TF; and LM is a linker moiety with a molecular weight less than 30,000 daltons; and wherein said compound inhibits TF activity with the proviso that the compound is not a compound having the formula of wtFVIIai-(DPTA-dim-FFR-cmk)-wtFVIIai or FVIIai(Q10E32)-(DPTA-dim-FFR-cmk)-FVIIai(Q10E32).

In a further aspect, the present invention relates to the use of a compound having the formula A-(LM)-D, wherein A and D are FVII polypeptides that binds to TF; and LM is a linker moiety with a molecular weight less than 30,000 daltons; and wherein said compound inhibits TF activity with the proviso that the compound is not a compound having the formula of wtFVIIai-(DPTA-dim-FFR-cmk)-wtFVIIai or FVIIai(Q10E32)-(DPTA-dim-FFR-cmk)-FVIIai(Q10E32) for the manufacture of a medicament for preventing or treating a TF related diseases or disorders.

In a further aspect, the present invention relates to a method for prevention or treatment of TF related diseases or disorders in a mammal, which method comprises administering to a mammal an effective amount of at least one compound having the formula A-(LM)-D, wherein A and D are FVII polypeptides that binds to TF; and LM is a linker moiety with a molecular weight less than 30,000 daltons; and wherein said compound inhibits TF activity with the proviso that the compound is not a compound having the formula of wtFVIIai-(DPTA-dim-FFR-cmk)-wtFVIIai or FVIIai(Q10E32)-(DPTA-dim-FFR-cmk)-FVIIai(Q10E32).

In a further aspect, the present invention relates to a compound having the formula A-(LM)-D, wherein A and D are FVII polypeptides that binds to TF; and LM is a linker moiety; and wherein said compound inhibits TF activity.

In a further aspect, the present invention relates to a compound having the formula A-(LM)-D, wherein A and D are FVII polypeptides that binds to TF; and LM is a linker moiety with a molecular weight less than 30,000 daltons; and wherein said compound inhibits TF activity.

In a further aspect, the present invention relates to a method for reducing TF activity, said method comprising contacting a TF expressing cell with a compound having the formula A-(LM)-D, wherein A and D are FVII polypeptides that binds to TF; and LM is a linker moiety with a molecular weight less than 30,000 daltons; and wherein said compound inhibits TF activity.

In a further aspect, the present invention relates to a pharmaceutical composition comprising an amount of the compound having the formula A-(LM)-D, wherein A and D are FVII polypeptides that binds to TF; and LM is a linker moiety with a molecular weight less than 30,000 daltons; and wherein said compound inhibits TF activity; and a pharmaceutically acceptable carrier or excipient.

In a further aspect, the present invention relates to a compound for use as a medicament having the formula A-(LM)-D, wherein A and D are FVII polypeptides that binds to TF; and LM is a linker moiety with a molecular weight less than 30,000 daltons; and wherein said compound inhibits TF activity.

In a further aspect, the present invention relates to the use of a compound having the formula A-(LM)-D, wherein A and D are FVII polypeptides that binds to TF; and LM is a linker moiety with a molecular weight less than 30,000 daltons; and wherein said compound inhibits TF activity for the manufacture of a medicament for preventing or treating a TF related diseases or disorders.

In a further aspect, the present invention relates to a method for prevention or treatment of TF related diseases or disorders in a mammal, which method comprises administering to a mammal an effective amount of at least one compound having the formula A-(LM)-D, wherein A and D are FVII polypeptides that binds to TF; and LM is a linker moiety with a molecular weight less than 30,000 daltons; and wherein said compound inhibits TF activity.

In one embodiment of the compound having the formula A-(LM)-D, the affinity of binding of said compound to TF is higher than the affinity of binding to TF of either A or D alone. In a specific embodiment the the affinity of binding of said compound to TF is 2 fold higher than the affinity of binding to TF of either A or D alone. In a specific embodiment the the affinity of binding of said compound to TF is 3 fold higher than the affinity of binding to TF of either A or D alone. In a specific embodiment the the affinity of binding of said compound to TF is 5 fold higher than the affinity of binding to TF of either A or D alone. In a specific embodiment the the affinity of binding of said compound to TF is 10 fold higher than the affinity of binding to TF of either A or D alone.

In a further embodiment of the invention, A and D of the compound having the formula A-(LM)-D are human recombinant FVIIa.

In a further embodiment of the invention, A and D of the compound having the formula A-(LM)-D are human recombinant FVIIai.

In a further embodiment of the invention, LM of the compound having the formula A-(LM)-D is a linker moiety with a molecular weight less than 20,000 daltons.

In a further embodiment of the invention, LM of the compound having the formula A-(LM)-D is a linker moiety with a molecular weight less than 10,000 daltons.

In a further embodiment of the invention, LM of the compound having the formula A-(LM)-D is a linker moiety with a molecular weight less than 5,000 daltons.

In a further embodiment of the invention, LM of the compound having the formula A-(LM)-D is a linker moiety with a molecular weight higher than 1,380 daltons.

In a further embodiment of the invention, LM of the compound having the formula A-(LM)-D is a linker moiety with a molecular weight higher than 1,400 daltons.

In a further embodiment of the invention, LM of the compound having the formula A-(LM)-D is a linker moiety with a molecular weight higher than 1,600 daltons.

In a further embodiment of the invention, LM of the compound having the formula A-(LM)-D is a linker moiety with a molecular weight higher than 1,800 daltons.

In a further embodiment of the invention, LM of the compound having the formula A-(LM)-D is a linker moiety with a molecular weight higher than 2,000 daltons.

In a further embodiment of the invention, LM of the compound having the formula A-(LM)-D is a linker moiety with a molecular weight higher than 3,000 daltons.

In a further embodiment of the invention, LM of the compound having the formula A-(LM)-D is a linker moiety with a molecular weight higher than 4,000 daltons.

In a further embodiment of the invention, LM of the compound having the formula A-(LM)-D is a linker moiety with a molecular weight higher than 5,000 daltons.

In a further embodiment of the invention, LM of the compound having the formula A-(LM)-D comprises an amino acid sequence. In a specific embodiment the compound having the formula A-(LM)-D only contains an amino acid sequence.

In a further embodiment of the invention, LM of the compound having the formula A-(LM)-D is a leucine zipper.

The terms “leucine zipper” or “LZ” as used herein, refers to any amino acid sequence with the pattern (H¹.P.P.H².P.P.P)ₙ where H are residues selected from Leu, Ile, Val or Asn, P are residues selected from Ser, Thr, Asn, Gin, Asp, Glu, Lys, Arg, His, Gly, Ala, Val, Leu, Ile, Phe and Tyr) and n≧2.

In a further embodiment of the invention, LM of the compound having the formula A-(LM)-D comprises a leucine zipper.

In a further embodiment of the invention, LM of the compound having the formula A-(LM)-D comprises an amino acid sequence (H¹.P.P.H².P.P.P)ₙ where H are residues selected from Leu, Ile, Val or Asn, P are residues selected from Ser, Thr, Asn, Gln, Asp, Glu, Lys, Arg, His, Gly, Ala, Val, Leu, Ile, Phe and Tyr) and n≧2.

In a further embodiment of the invention, LM of the compound having the formula A-(LM)-D comprises an amino acid sequence (H¹.P.P.H².P.P.P)ₙ where H are residues selected from Leu, Ile, Val, P are residues selected from Ser, Thr, Asn, Gin, Asp, Glu, Lys, Arg, His) and n≧4.

In a further embodiment of the invention, LM of the compound having the formula A-(LM)-D comprises an amino acid sequence (H¹.P.P.H².P.P.P)ₙ where H are Leu residues, P are residues selected from Ser, Thr, Asn, Gin, Asp, Glu, Lys, Arg, His) and n≧4.

In a further embodiment of the invention, LM of the compound having the formula A-(LM)-D comprises an amino acid sequence independently selected from the group consisting of SEQ ID NO: 5 and SEQ ID NO: 6.

In a further embodiment of the invention, LM of the compound having the formula A-(LM)-D comprises an amino acid sequence (Gly-Gly-Gly-Gly-Ser)n, wherein n is any integer from 1 to 10. In one embodiment n is 1-5. In a further embodiment n is 3.

In a further embodiment of the invention, the compound having the formula A-(LM)-D is not an immunoconjugate. It is to be understood, that the compounds of the present invention should preferably not mediate a cytolytic response of the immune system.

In a further embodiment of the invention, LM of the compound having the formula A-(LM)-D comprises a molecule selected from the group consisting of straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈ cycloalkyl, a 3 to 8-membered cyclic ring comprising carbon and at least one N, O or S atom in the ring, aryl, heteroaryl, amino acid, the structures optionally substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogeno, C₁₋₆ alkoxy, C₁₋₆ alkylthio, trifluoroalkoxy, alkoxycarbonyl, haloalkyl. The LM may be straight chained or branched and may contain one or more double or triple bonds. The LM may contain one or more heteroatoms like N,O or S. It is to be understood, that the LM can comprise more than one class of the groups described above, as well as being able to comprise more than one member within a class. Where the LM comprises more than one class of group, such LM is preferably obtained by joining different units via their functional groups. Methods for forming such bonds involve standard organic synthesis and are well known to those of ordinary skill in the art.

In a further embodiment of the invention, LM of the compound having the formula A-(LM)-D comprises a divalent chloromethyl ketone inhibitor consisting of two monomers independently selected from the group comprising Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone.

In a further embodiment of the invention, LM of the compound having the formula A-(LM)-D is a divalent FVIIa inhibitor.

In a further embodiment of the invention, LM of the compound having the formula A-(LM)-D comprises one FVIIa inhibitor.

In a further embodiment of the invention, LM of the compound having the formula A-(LM)-D comprises two FVIIa inhibitors.

In a further embodiment of the invention, LM of the compound having the formula A-(LM)-D is octanedioic acid bis-({1-[1-(1-chloroacetyl-4-guanidino-butylcarbamoyl)-2-phenyl-ethylcarbamoyl]-2-phenyl-ethyl}-amide.

In a further embodiment of the invention, LM of the compound having the formula A-(LM)-D is 10,12-docosadiyndioic acid bis-({1-[1-(1-chloroacetyl-4-guanidino-butylcarbamoyl)-2-phenyl-ethylcarbamoyl]-2-phenyl-ethyl}-amide).

In a further embodiment of the invention, LM of the compound having the formula A-(LM)-D is docosanedioic acid bis-({1-[1-(1-chloroacetyl-4-guanidino-butylcarbamoyl)-2-phenyl-ethylcarbamoyl]-2-phenyl-ethyl}-amide).

In a further embodiment of the invention, LM of the compound having the formula A-(LM)-D is icosanedioic acid bis-({1-[1-(1-chloroacetyl-4-guanidino-butylcarbamoyl)-2-phenyl-ethylcarbamoyl]-2-phenyl-ethyl}-amide).

In a further embodiment of the invention, LM of the compound having the formula A-(LM)-D is octadecadioic acid bis-({1-[1-(1-chloroacetyl-4-guanidino-butylcarbamoyl)-2-phenyl-ethylcarbamoyl]-2-phenyl-ethyl}-amide).

In a further embodiment of the invention, LM of the compound having the formula A-(LM)-D is 4,7,10,13-tetraoxahexadecanedioic acid bis-({1-[1-(1-chloroacetyl-4-guanidino-butylcarbamoyl)-2-phenyl-ethylcarbamoyl]-2-phenyl-ethyl}-amide).

In a further embodiment of the invention, LM of the compound having the formula A-(LM)-D is Ethyleneglycol-bis succinic acid bis-({1-[1-(1-chloroacetyl-4-guanidino-butylcarbamoyl)-2-phenyl-ethylcarbamoyl]-2-phenyl-ethyl}-amide).

In a further embodiment of the invention, LM of the compound having the formula A-(LM)-D is N-[3-(4-{3-[3-(1-{1-[1-(2-Chloroacetyl)-4-guanidinobutylcarbamoyl]-2-phenylethylcarbamoyl}-2-phenylethylcarbamoyl)propionylamino]propyl}piperazin-1-yl)-propyl]-succinamic acid -1-[1-(1-chloroacetyl-4-guanidino-butylcarbamoyl)-2-phenyl-ethylcarbamoyl]-2-phenyl-ethyl}-amide.

In a further embodiment of the invention, LM of the compound having the formula A-(LM)-D is Eicosanedioic acid bis-[(1-{1-[1-(2-chloro-acetyl)-4-guanidino-butylcarbamoyl]-2-phenyl-ethylcarbamoyl}-2-phenyl-ethyl)-amide].

In a further embodiment of the invention, LM of the compound having the formula A-(LM)-D is Pentanedioic acid bis-[(1-{1-[1-(2-chloro-acetyl)-4-guanidino-butylcarbamoyl]-2-phenyl-ethylcarbamoyl}-2-phenyl-ethyl)-amide].

In a further embodiment of the invention, LM of the compound having the formula A-(LM)-D is N,N′-bis-(1-{1-[1-(2-chloro-acetyl)-4-guanidino-butylcarbamoyl]-2-phenyl-ethylcarbamoyl}-2-phenylethyl)-oxalamide.

In a further embodiment, the pharmaceutical composition comprising an amount of the compound having the formula A-(LM)-D(LM)-D, wherein A and D are FVII polypeptides that binds to TF; and LM is a linker moiety with a molecular weight less than 30,000 daltons further contains a platelet aggregation inhibitor.

In a further embodiment of the invention, LM of the compound having the formula A-(LM)-D is a linker moiety with a molecular weight less than 20,000 daltons.

In a further embodiment of the invention, LM of the compound having the formula A-(LM)-D is a linker moiety with a molecular weight less than 10,000 daltons.

In a further embodiment of the invention, LM of the compound having the formula A-(LM)-D is a linker moiety with a molecular weight less than 5,000 daltons.

In a further embodiment of the invention, LM of the compound having the formula A-(LM)-D is a linker moiety with a molecular weight less than 2000 daltons.

In a further embodiment of the invention, LM of the compound having the formula A-(LM)-D is a linker moiety with a molecular weight less than 1000 daltons.

In a further embodiment LM is not [Bis-(2-{carboxymethyl-[(1-{1-[1-(2-chloro-acetyl)-4-guanidino-butylcarbamoyl]-2-phenyl-ethylcarbamoyl}-2-phenyl-ethylcarbamoyl)-methyl]-amino}-ethyl)-amino]-acetic acid.

In a further embodiment LM is not:

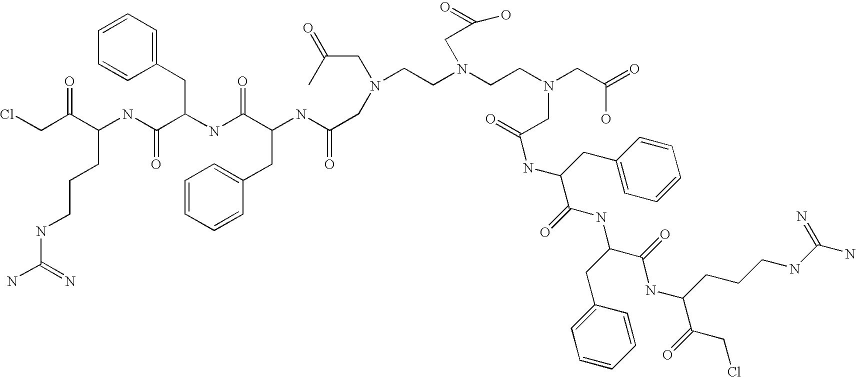

In a further embodiment LM is not:

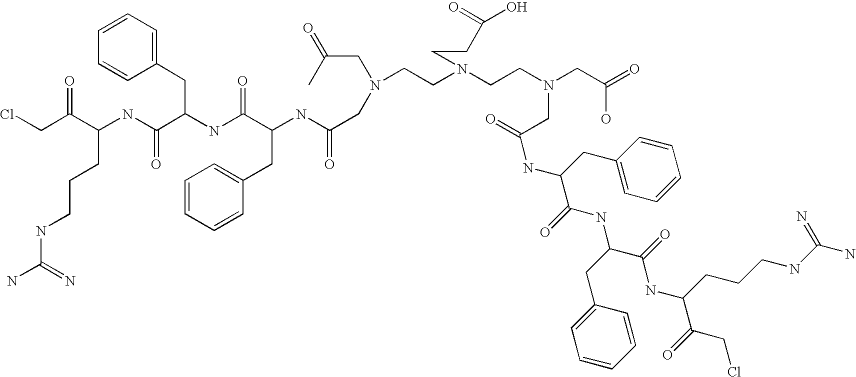

In a further embodiment of the invention the TF related diseases or disorders are deep venous thrombosis, arterial thrombosis, post surgical thrombosis, coronary artery bypass graft (CABG), percutaneous transdermal coronary angioplasty (PTCA), stroke, cancer, tumour metastasis, angiogenesis, ischemia/reperfusion, arthritis including rheumatoid arthritis, thrombolysis, arteriosclerosis and restenosis following angioplasty, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis.

The FVII polypeptides of the TF dimer antagonist are chemically bound to a molecular linker moiety. The LM allows the two FVII polypeptides to bind to the TF sites simultaneously. To accomplish this, the backbone must be long enough to span the distance between the TF sites, but also flexible enough to permit this binding to the second TF site. In other words, a suitable LM can assume a stable, extended secondary structure configuration, while remaining flexible and sufficiently soluble in aqueous or physiological systems.

By “linker moiety” (LM) or “backbone” is meant any biocompatible molecule functioning as a means to link the two FVII polypeptides. Each FVII polypeptide is linked to the molecular LM via a chemical bond, e.g. via an amide or peptide bond between an amino group of the LM and a carboxyl group, or its equivalent, of the FVII polypeptide, or vice versa. It is to be understood, that the LM may contain both covalent and non-covalent chemical bonds or mixtures thereof. By “flexible” is meant that the LM comprises a plurality of carbon-carbon σ bonds having free rotation about their axes, so as to allow the two FVII polypeptides to be separated by a distance suitable to bind two TF sites.

Suitable LMs, or backbones, comprise group(s) such as, but are not limited to, peptides; polynucleotides; sacharides including monosaccharides, di- and oligosaccharides, cyclodextrins and dextran; polymers including polyethylene glycol, polypropylene glycol, polyvinyl alcohol, hydrocarbons, polyacrylates and amino-, hydroxy-, thio- or carboxy-functionalised silicones, other biocompatible material units; and combinations thereof. Such LM materials described above are widely commercially available or obtainable via synthetic organic methods commonly known to those skilled in the art.

The LM may, for example, be selected among the following structures:

straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈ cycloalkyl, a 3 to 8-membered cyclic ring comprising carbon and at least one N, O or S atom in the ring, aryl, heteroaryl, amino acid, the structures optionally substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido , halogeno, C₁₋₆ alkoxy, C₁₋₆ alkylthio, trifluoroalkoxy, alkoxycarbonyl, haloalkyl. The LM may be straight chained or branched and may contain one or more double or triple bonds. The LM may contain one or more heteroatoms like N,O or S. It is to be understood, that the LM can comprise more than one class of the groups described above, as well as being able to comprise more than one member within a class. Where the LM comprises more than one class of group, such LM is preferably obtained by joining different units via their functional groups. Methods for forming such bonds involve standard organic synthesis and are well known to those of ordinary skill in the art.

It should be noted that peptides, proteins and amino acids as used herein can comprise or refer to “natural”, i.e., naturally occurring amino acids as well as “non classical” D-amino acids including, but not limited to, the D-isomers of the common amino acids, α-isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, Nα-methyl amino acids, and amino acid analogues in general. In addition, the amino acids can include Abu, 2-amino butyric acid; γ-Abu, 4-aminobutyric acid; ε-Ahx, 6-aminohexanoic acid; Aib, 2-amino-isobutyric acid; β-Ala, 3-aminopropionic acid; Orn, ornithine; Hyp, trans-hydroxyproline; Nle, norleucinw; Nva, norvaline.

The three-letter indication “GLA” as used herein means 4-carboxyglutamic acid (γ-carboxyglutamate).

By “combinations thereof” is meant that the LM can comprise more than one class of the groups described above, as well as being able to comprise more than one member within a class. Where the LM comprises more than one class of group, such LM is preferably obtained by joining different units via their functional groups. Methods for forming such bonds involve standard organic synthesis and are well known to those of ordinary skill in the art.

In order to select a suitable linker moiety, the FVII polypeptides are coupled to different ferent LM's and screened in a TF activity assay.

The LM can comprise functional groups, such as, for example hydroxy, oxo, amino, C₁₋₄ monoalkylamino, acylamino, sulfonyl, carboxy, carboxamido, halogeno, C₁₋₆ alkoxy, C₁₋₆ alkylthio, trifluoroalkoxy, alkoxycarbonyl, or haloalkyl groups. The LM can also comprise charged functional groups, such as for example, ammonium groups or carboxylate groups.

The charged functional groups can provide TF dimer antagonists with sufficient solubility in aqueous or physiological systems, provide reactive sites for ionic bonding with other species, and enhance their avidity to other members of the TF/FVIIa/FXa complex. It is within the purview of one of skill in the art to select a particular acid, and concentration thereof, to confer optimal solubility and avidity properties to the TF dimer antagonists. Preferably, the total amount of charged functional groups are minimised so as to maximise the TF dimer antagonists specificity for TF sites, but not so as to significantly decrease solubility.

The FVII polypeptides are situated on the LM such that the distance between them is sufficient to allow the FVII polypeptides to bind to two TF molecules. Because the LM of the TF dimer antagonist is flexible, the FVII polypeptides are capable of assuming a conformation that allows the FVII polypeptides to bind to two TF molecules. Such a distance between the FVII polypeptides can be measured, or predicted theoretically, by any method known in the art, e.g. molecular modelling. Molecular modelling programs that can be used are commonly known and available in the art.

It is to be understood that the two FVII polypeptides in a TF dimer antagonist can be the same or different.

The LM can further comprise a FVIIa inhibitor.

By a “FVIIa inhibitor” is meant a substance binding to FVIIa and decreasing or preventing the FVIIa-catalysed conversion of FX to FXa. A FVIIa inhibitor may be identified as a substance A, which reduces the amidolytic activity by at least 50% at a concentration of substance stance A at 400 μM in the FVIIa amidolytic assay described by Persson et al. (Persson et al., J. Biol. Chem. 272: 19919-19924 (1997)). Preferred are substances reducing the amidolytic activity by at least 50% at a concentration of substance A at 300 μM; more preferred are substances reducing the amidolytic activity by at least 50% at a concentration of substance A at 200 μM.

The “FVIIa inhibitor” may be selected from any one of several groups of FVIIa directed inhibitors. Such inhibitors are broadly categorised for the purpose of the present invention into i) inhibitors which reversibly bind to FVIIa and are cleavable by FVIIa, ii) inhibitors which reversibly bind to FVIIa but cannot be cleaved, and iii) inhibitors which irreversibly bind to FVIIa. For a review of inhibitors of serine proteases see Proteinase Inhibitors (Research Monographs in cell and Tissue Physiology; v. 12) Elsevier Science Publishing Co., Inc., New York (1990).

The FVIIa inhibitor moiety may also be an irreversible FVIIa serine protease inhibitor. Such irreversible active site inhibitors generally form covalent bonds with the protease active site. Such irreversible inhibitors include, but are not limited to, general serine protease inhibitors such as peptide chloromethylketones (see, Williams et al., J. Biol. Chem. 264:7536-7540 (1989)) or peptidyl cloromethanes; azapeptides; acylating agents such as various guanidinobenzoate derivatives and the 3-alkoxy-4-chloroisocoumarins; sulphonyl fluorides such as phenylmethylsulphonylfluoride (PMSF); diisopropylfluorophosphate (DFP); tosylpropylchloromethyl ketone (TPCK); tosyllysylchloromethyl ketone (TLCK); nitrophenyl-sulphonates and related compounds; heterocyclic protease inhibitors such as isocoumarines, and coumarins.

Examples of peptidic irreversible FVIIa inhibitors include, but are not limited to,

Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone.

Examples of FVIIa inhibitors also include benzoxazinones or heterocyclic analogues thereof such as described in PCT/DK99/00138.

Examples of other FVIIa inhibitors include, but are not limited to, small peptides such as for example Phe-Phe-Arg, D-Phe-Phe-Arg, Phe-Phe-Arg, D-Phe-Phe-Arg, Phe-Pro-Arg, D-Phe-Pro-Arg, Phe-Pro-Arg, D-Phe-Pro-Arg, L- and D-Glu-Gly-Arg; peptidomimetics; benzamidine systems; heterocyclic structures substituted with one or more amidino groups; aromatic or heteroaromatic systems substituted with one or more C(═NH)NHR groups in which R is H, C₁₋₃ alkyl, OH or a group which is easily split of in vivo.

The terms “C₁₋₅₀-alkyl” or “C₁₋₅₀-alkanediyl” as used herein, refers to a straight or branched, saturated or unsaturated hydrocarbon chain having from one to 50 carbon atoms.

The terms “C₂₋₅₀-alkenyl” or “C₂₋₅₀-alkenediyl” as used herein, refers to an unsaturated branched or straight hydrocarbon chain having from 2 to 50 carbon atoms and at least one double bond.

The terms “C₂₋₅₀-alkynyl” or “C₂₋₅₀-alkynediyl” as used herein, refers to an unsaturated branched or straight hydrocarbon chain having from 2 to 50 carbon atoms and at least one triple bond. The C₁₋₅₀-alkyl residues include aliphatic hydrocarbon residues, unsaturated aliphatic hydrocarbon residues, alicyclic hydrocarbon residues. Examples of a C₁₋₅₀-alkyl within this definition include but are not limited to decanyl, hexadecanyl, octadecanyl, nonadecanyl, icosanyl, docosanyl, tetracosanyl, triacontanyl, decanediyl, hexadecanediyl, octadecanediyl, nonadecanediyl, icosanediyl, docosanediyl, tetracosanediyl, triacontanediyl, The term C₃₋₈-cycloalkyl means an alicyclic hydrocarbon residue including saturated alicyclic hydrocarbon residues having 3 to 8 carbon atoms such as cyclopropyl, cyclobutyl, cyclopentyl, cyclohexyl; and C₅₋₆ unsaturated alicyclic hydrocarbon residues having 5 to 6 carbon atoms such as 1-cyclopentenyl, 2-cyclopentenyl, 3-cyclopentenyl, 1-cyclohexenyl, 2-cyclohexenyl, 3-cyclohexenyl.

The term “C₁₋₆-alkoxy” as used herein, alone or in combination, refers to a straight or branched monovalent substituent comprising a C₁₋₆-alkyl group linked through an ether oxygen having its free valence bond from the ether oxygen and having 1 to 6 carbon atoms e.g. methoxy, ethoxy, propoxy, isopropoxy, butoxy, pentoxy.

The term “C₁₋₆-alkylthio” as used herein, alone or in combination, refers to a straight or branched monovalent substituent comprising a C₁₋₆-alkyl group linked through an thioether sulfur atom having its free valence bond from the thioether sulfur and having 1 to 6 carbon atoms.

The terms “aryl” and “heteroaryl” as used herein refers to an aryl which can be optionally substituted or a heteroaryl which can be optionally substituted and includes phenyl, biphenyl, indene, fluorene, naphthyl (1-naphthyl, 2-naphthyl), anthracene (1-anthracenyl, 2-anthracenyl, 3-anthracenyl), thiophene (2-thienyl, 3-thienyl), furyl (2-furyl, 3-furyl), indolyl, oxadiazolyl, isoxazolyl, quinazolin, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), iso-quinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl).

The invention also relates to partly or fully saturated analogues of the ring systems mentioned above.

The terms “C₁₋₄ monoalkylamino” and “C₁₋₄ dialkylamino” refer to an amino group having one or both of its hydrogens independently replaced by an alkyl group having 1 to 4 carbon atoms, alkyl being defined above, such as methylamino, dimethylamino, N-ethyl-N-methylamino, ethylamino, diethylamino, propylamino, dipropylamino, N-(n-butyl)-N-methylamino, n-butylamino, di(n-butyl)amino, sec-butylamino, t-butylamino, and the like.

The terms “acyl” or “carboxy” refer to a monovalent substituent comprising a C₁₋₆-alkyl group linked through a carbonyl group; such as e.g. acetyl, propionyl, butyryl, isobutyryl, pivaloyl, valeryl, and the like.

The term “acylamino” refers to the group C₁₋ₙC(═O)NH—.

The term “carboxamido” refers to the group —C(═O)NHC₁₋ₙ.

The term “trifluoroalkoxy” refers to an C₁₋₆ alkoxy group as defined above having three of its hydrogen atoms bonded to one or more of the carbon atoms replaced by fluor atoms, such as (CF_{3)O—, (CF}_{3)CH}₂O—.

The term “alkoxycarbonyl” refers to the group —C(═O)(R) where R is an C₁₋₆ alkoxy group as defined above. The term “C₁₋₆-alkoxycarbonyl” as used herein refers to a monovalent substituent comprising a C₁₋₆-alkoxy group linked through a carbonyl group; such as e.g. methoxycarbonyl, carbethoxy, propoxycarbonyl, isopropoxycarbonyl, n-butoxycarbonyl, secbutoxycarbonyl, tert-butoxycarbonyl, 3-methylbutoxycarbonyl, n-hexoxycarbonyl and the like.

The term “leaving group” as used herein includes, but is not limited to, halogen, sulfonate or an acyl group. Suitable leaving groups will be known to a person skilled in the art.

“Halogen” refers to fluorine, chlorine, bromine, and iodine. “Halo” refers to fluoro, chloro, bromo and iodo.

“Optional” or “optionally” means that the subsequently described event or circumstances may or may not occur, and that the description includes instances where said event or circumstance occur and instances in which is does not. For example, “aryl . . . optionally substituted” means that the aryl may or may not be substituted and that the description includes both unsubstituted aryls and aryls wherein there is substitution.

The LM comprising FVIIa inhibitors to be used in the preparation of a TF dimer antagonist may be prepared by the following methods. In the following methods FVIIa inhibitors are designated the letter Fₙ, wherein n is 1 or 2 to indicate that the LM may comprise two different FVIIa inhibitors.

Method 1.

LM comprising FVIIa inhibitors is prepared by reacting F₁—B—X, in which X is a functional group capable of reacting with structures F₂—Y, in which Y is a functional group, by means of normal coupling reactions using coupling reagents known by the person skilled in the art.

Method 2.

LM comprising FVIIa inhibitors may be prepared by reaction between F₁—B-Z, in which Z is a leaving group and F₂—W in which W is a nucleofile. Examples of leaving groups are halogens, sulfonates, phosphonates. Examples of nucleofiles are hydroxy , amino, N-substituted amino, and carbanions.

Method 3.

LM comprising FVIIa inhibitors may be prepared by reaction between F₂—B-Z, in which Z is a leaving group, and F₁—W, in which W is a nucleofile. Examples of leaving groups are halogens, sulfonates, phosphonates. Examples of nucleofiles are hydroxy, amino, N-substituted amino, and carbanions.

Method 4.

The linker B can be reacted with structures F₁ and F₂ connected to a solid phase surface using methods well known in the art. This approach is especially valuable when the structures F₁ and F₂ are identical, the method here resulting in an marked increase in the yield due to the optimal distance between the solid phase coupled reagents and the linker size.

Method 5.

LM comprising FVIIa inhibitors may be prepared by a sequence of reactions through which F₁ or F₂ firstly are reacted with the activated linker moiety forming F₁—B, respectively F₂—B moieties and subsequently the formed product is reacted with F₂, respectively F₁ moiety. The actual bond formation taking place through reaction on functional groups or derivatives or leaving groups /nucleofiles as described under methods 1-3.

The reaction can be carried out in solution phase or on a solid phase support using the procedures known by the person skilled in the art.

Formation of FVII Dimer by DNA Technology

While the use of active site directed reagents represents one approach to producing TF dimeric antagonists, this can also be achieved by adding peptide tags on the FVII poly-peptides with the ability to specifically join two or more FVII polypeptides through tight non-covalent interactions. The perhaps most suitable tag for achieving this is via the leucine zipper (LZ) domain which is a naturally occurring element capable of mediating tight protein-protein interactions. The LZ's are present in many gene regulatory proteins, such as: The CCATT-box and enhancer binding protein (C/EBP), The cAMP response element (CRE) binding proteins (CREB, CRE-BP1, ATFs), The Jun/AP1 family of transcription factors, The yeast general control protein GCN4, The fos oncogene, and the fos-related proteins fra-1 and fos B, The C-myc, L-myc and N-myc oncogenes, The octamer-binding transcription factor 2 (Oct-2/OTF-2).

Structurally the LZ consist of a periodic repetition of leucine or other hydrophobic residues at every seventh position over a distance covering typically eight helical turns according to the pattern (H¹.P.P.H².P.P.P)ₙ where H is selected from Leu, Ile, Val or Asn and n≧2, typically n≧4. The segments containing these periodic arrays of leucine residues exist in an amphipatic alpha-helical conformation. The leucine side chains extending from one alpha-helix tagged on one FVII polypeptide interact with those from a similar alpha helix tagged on a second FVII polypeptide, facilitating dimerization; the structure formed by cooperation of these two regions forms a coiled coil. The structural data available on the LZ's indicate that the dimeric form exists in a parallel conformation, i.e., the N-termini of both helixes are at the same end of the dimer. This is important as it may provide a presentation of dimerized FVII polypeptides in which the TF binding domains both are in an orientation facilitating binding. An additional feature of the LZ motif is that specific mutations at the two hydrophobic positions in the peptide allows the user to switch between di-, tri- or tetrameric conformation of the target protein. In the more complex oligomeric forms of LZ provides for different orientation of the individual elements depending on the composition of the individual binding partners (Harbury, P. B. et al. Science (1993) 262: 1401-1407).

| | H¹ | H² |
|---|---|---|
| Dimer | Ile or | Leu |
| | Val | |
| Trimer | Ile | Ile |
| Tetramer | Leu | Ile |

The perhaps best studied LZ is that of the yeast general control protein GCN4, however, since immunogenicity may be a concern, a humanized product or one of the human equivalents may be used. Thus, in a preferred embodiment the LZ from human ATF7 or ATF4 and derivatives thereof is used. The actual fusion protein may be produced using conventional molecular biology methods known to those skilled in the art, including PCR or primer-dimer cassettes. The tag will be introduced C-terminal to the coding sequence of the FVII polypeptide and will be preceded by a short spacer segment (Gly-Ser-Ala) which serves to provide a degree of conformational flexibility, thus, favoring formation of the desired oligomers (other linker segments can be imagined, indeed a longer spacer may be required to avoid structural clashes upon oligomerization). Thus, one example of a TF Dimeric antagonist according to the invention could be the naturally formed dimer of a FVII-Gly-Ser-Ala-LZ construct. The FVII-LZ fusion protein are produced as described for other FVII polypeptides.

In the present specification, amino acids are represented using abbreviations, as indicated in table 1, approved by IUPAC-IUB Commission on Biochemical Nomenclature (CBN). Amino acid and the like having isomers represented by name or the following abbreviations are in natural L-form unless otherwise indicated. Further, the left and right ends of an amino acid sequence of a peptide are, respectively, the N- and C-termini unless otherwise specified.

TABLE 1
Abbreviations for amino acids:

| Amino acid | Tree-letter code | One-letter code |
|---|---|---|
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gln | Q |
| Asparagine | Asn | N |
| Glutamic Acid | Glu | E |
| Aspartic Acid | Asp | D |
| Serine | Ser | S |
| Threonine | Thr | T |

The invention also relates to a method of preparing human FVII polypeptides as mentioned above. The human FVII polypeptides are preferably produced by recombinant DNA techniques. To this end, DNA sequences encoding human FVII may be isolated by preparing a genomic or cDNA library and screening for DNA sequences coding for all or part of the protein by hybridization using synthetic oligonucleotide probes in accordance with standard techniques (cf. Sambrook et al., Molecular Cloning: A Laboratory Manual, Cold Spring Harbor Laboratory, Cold Spring Harbor, N.Y., 1989). For the present purpose, the DNA sequence encoding the protein is preferably of human origin, i.e. derived from a human genomic DNA or cDNA library.

The DNA sequences encoding the human FVII polypeptides may also be prepared synthetically by established standard methods, e.g. the phosphoamidite method described by Beaucage and Caruthers, Tetrahedron Letters 22 (1981), 1859-1869, or the method described by Mafthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

The DNA sequences may also be prepared by polymerase chain reaction using specific primers, for instance as described in U.S. Pat. No. 4,683,202, Saiki et al., Science 239 (1988), 487-491, or Sambrook et al., supra.

The DNA sequences encoding the human FVII polypeptides are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the human FVII polypeptides is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

The promoter may be any DNA sequence, which shows transcriptional activity in the host cell of choice and may be derived from genes encoding proteins either homologous or heterologous to the host cell.

Examples of suitable promoters for directing the transcription of the DNA encoding the human FVII polypeptide in mammalian cells are the SV40 promoter (Subramani et al., Mol. Cell Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI 1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nindulans acetamidase. Preferred are the TAKA-amylase and gluA promoters. Suitable promoters are mentioned in, e.g. EP 238 023 and EP 383 779.

The DNA sequences encoding the human FVII polypeptides may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. The vector may also contain a set of RNA splice sites located downstream from the promoter and upstream from the insertion site for the FVII sequence itself. Preferred RNA splice sites may be obtained from adenovirus and/or immunoglobulin genes. Also contained in the expression vectors is a polyadenylation signal located downstream of the insertion site. Particularly preferred polyadenylation signals include the early or late polyadenylation signal from SV40 (Kaufman and Sharp, ibid.), the polyadenylation signal from the adenovirus 5 E1B region, the human growth hormone gene terminator (DeNoto et al. Nuc. Acids Res. 9:3719-3730, 1981) or the polyadenylation signal from the human FVII gene or the bovine FVII gene. The expression vectors may also include a noncoding viral leader sequence, such as the adenovirus 2 tripartite leader, located between the promoter and the RNA splice sites; and enhancer sequences, such as the SV40 enhancer.

The recombinant vector may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD or sC.

To direct the human FVII polypeptides of the present invention into the secretory pathway of the host cells, a secretory signal sequence (also known as a leader sequence, prepro sequence or pre sequence) may be provided in the recombinant vector. The secretory signal sequence is joined to the DNA sequences encoding the human FVII polypeptides in the correct reading frame. Secretory signal sequences are commonly positioned 5′ to the DNA sequence encoding the peptide. The secretory signal sequence may be that, normally associated with the protein or may be from a gene encoding another secreted protein.

For secretion from yeast cells, the secretory signal sequence may encode any signal peptide, which ensures efficient direction of the expressed human FVII polypeptides into the secretory pathway of the cell. The signal peptide may be naturally occurring signal peptide, or a functional part thereof, or it may be a synthetic peptide. Suitable signal peptides have been found to be the a-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the human FVII polypeptides. The function of the leader peptide is to allow the expressed peptide to be directed from the endoplasmic reticulum to the Golgi apparatus and further to a secretory vesicle for secretion into the culture medium (i.e. exportation of the human FVII polypeptides across the cell wall or at least through the cellular membrane into the periplasmic space of the yeast cell). The leader peptide may be the yeast alpha-factor leader (the use of which is described in e.g. U.S. Pat. No. 4,546,082, U.S. Pat. No. 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the human FVII polypeptides, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells.

After the cells have taken up the DNA, they are grown in an appropriate growth medium, typically 1-2 days, to begin expressing the gene of interest. As used herein the term “appropriate growth medium” means a medium containing nutrients and other components required for the growth of cells and the expression of the human FVII polypeptides of interest. Media generally include a carbon source, a nitrogen source, essential amino acids, essential sugars, vitamins, salts, phospholipids, protein and growth factors. For production of gamma-carboxylated proteins, the medium will contain vitamin K, preferably at a concentration of about 0.1 μg/ml to about 5 μg/ml. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of the human FVII polypeptide of interest.

The host cell into which the DNA sequences encoding the human FVII polypeptides is introduced may be any cell, which is capable of producing the post-translational modified human FVII polypeptides and includes yeast, fungi and higher eucaryotic cells.

Examples of mammalian cell lines for use in the present invention are the COS-1 (ATCC CRL 1650), baby hamster kidney (BHK) and 293 (ATCC CRL 1573; Graham et al., J. Gen. Virol. 36:59-72, 1977) cell lines. A preferred BHK cell line is the tk⁻ts13 BHK cell line (Waechter and Baserga, Proc. Natl. Acad. Sci. USA 79:1106-1110, 1982, incorporated herein by reference), hereinafter referred to as BHK 570 cells. The BHK 570 cell line has been deposited with the American Type Culture Collection, 12301 Parklawn Dr., Rockville, Md. 20852, under ATCC accession number CRL 10314. A tk⁻ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220, 1980).

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding the human FVII polypeptides may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F. oxysporum may, for instance, be carried out as described by Malardier et al., 1989, Gene 78: 147-156. The transformation of Trichoderma spp. may be performed for instance as described in EP 244 234.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting expression of the human FVII polypeptide after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The human FVII polypeptide produced by the cells may then be recovered from the culture medium by conventional procedures including separating the host cells from the medium by centrifugation or filtration, precipitating the proteinaqueous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of polypeptide in question.

For the preparation of recombinant human FVII polypeptides, a cloned wild-type FVII DNA sequence is used. This sequence may be modified to encode a desired FVII variant. The complete nucleotide and amino acid sequences for human FVII are known. See U.S. Pat. No. 4,784,950, which is incorporated herein by reference, where the cloning and expression of recombinant human FVII is described. The bovine FVII sequence is described in Takeya et al., J. Biol. Chem, 263:14868-14872 (1988), which is incorporated by reference herein.

The amino acid sequence alterations may be accomplished by a variety of techniques. Modification of the DNA sequence may be by site-specific mutagenesis. Techniques for site-specific mutagenesis are well known in the art and are described by, for example, Zoller and Smith (DNA 3:479-488, 1984). Thus, using the nucleotide and amino acid sequences of FVII, one may introduce the alterations of choice.

DNA sequences for use within the present invention will typically encode a pre-pro peptide at the amino-terminus of the FVII protein to obtain proper post-translational processing (e.g. gamma-carboxylation of glutamic acid residues) and secretion from the host cell. The pre-pro peptide may be that of FVII or another vitamin K-dependent plasma protein, such as factor IX, factor X, prothrombin, protein C or protein S. As will be appreciated by those skilled in the art, additional modifications can be made in the amino acid sequence of FVII where those modifications do not significantly impair the ability of the protein to act as a coagulation factor. For example, FVII in the catalytic triad can also be modified in the activation cleavage site to inhibit the conversion of zymogen FVII into its activated two-chain form, as generally described in U.S. Pat. No. 5,288,629, incorporated herein by reference.

Within the present invention, transgenic animal technology may be employed to produce the human FVII polypeptide. It is preferred to produce the proteins within the mammary glands of a host female mammal. Expression in the mammary gland and subsequent secretion of the protein of interest into the milk overcomes many difficulties encountered in isolating proteins from other sources. Milk is readily collected, available in large quantities, and well characterized biochemically. Furthermore, the major milk proteins are present in milk at high concentrations (typically from about 1 to 15 g/l). From a commercial point of view, it is clearly preferable to use as the host a species that has a large milk yield. While smaller animals such as mice and rats can be used (and are preferred at the proof of principle stage), within the present invention it is preferred to use livestock mammals including, but not limited to, pigs, goats, sheep and cattle. Sheep are particularly preferred due to such factors as the previous history of transgenesis in this species, milk yield, cost and the ready availability of equipment for collecting sheep milk. See WIPO Publication WO 88/00239 for a comparison of factors influencing the choice of host species. It is generally desirable to select a breed of host animal that has been bred for dairy use, such as East Friesland sheep, or to introduce dairy stock by breeding of the transgenic line at a later date. In any event, animals of known, good health status should be used.

To obtain expression in the mammary gland, a transcription promoter from a milk protein gene is used. Milk protein genes include those genes encoding caseins (see U.S. Pat. No. 5,304,489, incorporated herein by reference), beta-lactoglobulin, alpha-lactalbumin, and whey acidic protein. The beta-lactoglobulin (BLG) promoter is preferred. In the case of the ovine beta-lactoglobulin gene, a region of at least the proximal 406 bp of 5′ flanking sequence of the gene will generally be used, although larger portions of the 5′ flanking sequence, up to about 5 kbp, are preferred, such as about 4.25 kbp DNA segment encompassing the 5′ flanking promoter and non-coding portion of the beta-lactoglobulin gene. See Whitelaw et al., Biochem J. 286: 31-39 (1992). Similar fragments of promoter DNA from other species are also suitable.

Other regions of the beta-lactoglobulin gene may also be incorporated in constructs, as may genomic regions of the gene to be expressed. It is generally accepted in the art that constructs lacking introns, for example, express poorly in comparison with those that contain such DNA sequences (see Brinster et al., Proc. Natl. Acad. Sci. USA 85: 836-840 (1988); Palmiter et al., Proc. Natl. Acad. Sci. USA 88: 478-482 (1991); Whitelaw et al., Transgenic Res. 1: 3-13 (1991); WO 89/01343; and WO 91/02318, each of which is incorporated herein by reference). In this regard, it is generally preferred, where possible, to use genomic sequences containing all or some of the native introns of a gene encoding the protein or polypeptide of interest, thus the further inclusion of at least some introns from, e.g, the beta-lactoglobulin gene, is preferred. One such region is a DNA segment which provides for intron splicing and RNA polyadenylation from the 3′ non-coding region of the ovine beta-lactoglobulin gene. When substituted for the natural 3′ non-coding sequences of a gene, this ovine beta-lactoglobulin segment can both enhance and stabilize expression levels of the protein or polypeptide of interest. Within other embodiments, the region surrounding the initiation ATG of the sequence encoding the human FVII polypeptide is replaced with corresponding sequences from a milk specific protein gene. Such replacement provides a putative tissue-specific initiation environment to enhance expression. It is convenient to replace the entire pre-pro sequence of the human FVII polypeptide and 5′ non-coding sequences with those of, for example, the BLG gene, although smaller regions may be replaced.

For expression of a human FVII polypeptide in transgenic animals, a DNA segment encoding the human FVII polypeptide is operably linked to additional DNA segments required for its expression to produce expression units. Such additional segments include the above-mentioned promoter, as well as sequences which provide for termination of transcription and polyadenylation of mRNA. The expression units will further include a DNA segment encoding a secretory signal sequence operably linked to the segment encoding the human FVII polypeptide. The secretory signal sequence may be a native secretory signal sequence of the human FVII polypeptide or may be that of another protein, such as a milk protein. See, for example, von Heinje, Nuc. Acids Res. 14: 4683-4690 (1986); and Meade et al., U.S. Pat. No. 4,873,316, which are incorporated herein by reference.

Construction of expression units for use in transgenic animals is conveniently carried out by inserting a sequence encoding the human FVII polypeptide into a plasmid or phage vector containing the additional DNA segments, although the expression unit may be constructed by essentially any sequence of ligations. It is particularly convenient to provide a vector containing a DNA segment encoding a milk protein and to replace the coding sequence for the milk protein with that of the human FVII polypeptide, thereby creating a gene fusion that includes the expression control sequences of the milk protein gene. In any event, cloning of the expression units in plasmids or other vectors facilitates the amplification of the human FVII polypeptide. Amplification is conveniently carried out in bacterial (e.g. E. coli) host cells, thus the vectors will typically include an origin of replication and a selectable marker functional in bacterial host cells.

The expression unit is then introduced into fertilized eggs (including early-stage embryos) of the chosen host species. Introduction of heterologous DNA can be accomplished by one of several routes, including microinjection (e.g. U.S. Pat. No. 4,873,191), retroviral infection (Jaenisch, Science 240: 1468-1474 (1988)) or site-directed integration using embryonic stem (ES) cells (reviewed by Bradley et al., Bio/Technology 10: 534-539 (1992)). The eggs are then implanted into the oviducts or uteri of pseudopregnant females and allowed to develop. Offspring carrying the introduced DNA in their germ line can pass the DNA on to their progeny in the normal, Mendelian fashion, allowing the development of transgenic herds.

General procedures for producing transgenic animals are known in the art. See, for example, Hogan et al., Manipulating the Mouse Embryo: A Laboratory Manual, Cold Spring Harbor Laboratory, 1986; Simons et al., Bio/Technology 6: 179-183 (1988); Wall et al., Biol. Reprod. 32: 645-651 (1985); Buhler et al., Bio/Technology 8: 140-143 (1990); Ebert et al., Bio/Technology 9: 835-838 (1991); Krimpenfort et al., Bio/Technology 9: 844-847 (1991); Wall et al., J. Cell. Biochem. 49:113-120 (1992); U.S. Pat. Nos. 4,873,191 and 4,873,316; WIPO publications WO 88/00239, WO 90/05188, WO 92/11757; and GB 87/00458, which are incorporated herein by reference. Techniques for introducing foreign DNA sequences into mammals and their germ cells were originally developed in the mouse. See, e.g., Gordon et al., Proc. Natl. Acad. Sci. USA 77: 7380-7384 (1980); Gordon and Ruddle, Science 214: 1244-1246 (1981); Palmiter and Brinster, Cell 41: 343-345 (1985); and Brinsteret et al., Proc. Natl. Acad. Sci. USA 82: 4438-4442 (1985). These techniques were subsequently adapted for use with larger animals, including livestock species (see e.g., WIPO publications WO 88/00239, WO 90/05188, and WO 92/11757; and Simons et al., Bio/Technology 6: 179-183 (1988). To summarize, in the most efficient route used to date in the generation of transgenic mice or livestock, several hundred linear molecules of the DNA of interest are injected into one of the pro-nuclei of a fertilized egg according to established techniques. Injection of DNA into the cytoplasm of a zygote can also be employed. Production in transgenic plants may also be employed. Expression may be generalized or directed to a particular organ, such as a tuber. See, Hiatt, Nature 344:469-479 (1990); Edelbaum et al., J. Interferon Res. 12:449-453 (1992); Sijmons et al., Bio/Technology 8:217-221 (1990); and European Pat. Office Publication EP 255,378.

FVII produced according to the present invention may be purified by affinity chromatography on an anti-FVII antibody column. It is preferred that the immunoadsorption column comprise a high-specificity monoclonal antibody. The use of calcium-dependent monoclonal antibodies, as described by Wakabayashi et al., J. Biol. Chem, 261:11097-11108, (1986) and Thim et al., Biochem. 27: 7785-7793, (1988), incorporated by reference herein, is particularly preferred. Additional purification may be achieved by conventional chemical purification means, such as high performance liquid chromatography. Other methods of purification, including barium citrate precipitation, are known in the art, and may be applied to the purification of the FVII described herein (see, generally, Scopes, R., Protein Purification, Springer-Verlag, N.Y., 1982). Substantially pure FVII of at least about 90 to 95% homogeneity is preferred, and 98 to 99% or more homogeneity most preferred, for pharmaceutical uses. Once purified, partially or to homogeneity as desired, the FVII may then be used therapeutically.

Conversion of single-chain FVII to active two-chain FVIIa may be achieved using factor XIIa as described by Hedner and Kisiel (1983, J. Clin. Invest. 71: 1836-1841), or with other proteases having trypsin-like specificity (Kisiel and Fujikawa, Behring Inst. Mitt. 73: 29-42, 1983). Alternatively FVII may be autoactivated by passing it through an ion-exchange chromatography column, such as mono Q.RTM. (Pharmacia Fire Chemicals) or the like (Bjoern et al., 1986, Research Disclosures 269:564-565). The FVII molecules of the present invention and pharmaceutical compositions thereof are particularly useful for administration to humans to treat a variety of conditions involving intravascular coagulation.

The compounds of the present invention may have one or more asymmetric centres and it is intended that stereoisomers (optical isomers), as separated, pure or partially purified stereoisomers or racemic mixtures thereof are included in the scope of the invention.

Within the present invention, the TF dimer antagonist may be prepared in the form of pharmaceutically acceptable salts, especially acid-addition salts, including salts of organic acids and mineral acids. Examples of such salts include salts of organic acids such as formic acid, fumaric acid, acetic acid, propionic acid, glycolic acid, lactic acid, pyruvic acid, oxalic acid, succinic acid, malic acid, tartaric acid, citric acid, benzoic acid, salicylic acid and the like. Suitable inorganic acid-addition salts include salts of hydrochloric, hydrobromic, sulphuric and phosphoric acids and the like. Further examples of pharmaceutically acceptable inorganic or organic acid addition salts include the pharmaceutically acceptable salts listed in Journal of Pharmaceutical Science, 66, 2 (1977) which are known to the skilled artisan.

Also intended as pharmaceutically acceptable acid addition salts are the hydrates which the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of this invention may form solvates with standard low molecular weight solvents using methods known to the skilled artisan.

The TF dimer antagonist of the invention are useful for the preparation of a pharmaceutical composition for the treatment of or prophylaxis of thrombotic or coagulopathic related diseases or disorders including vascular diseases and inflammatory responses. This includes, but are not limited to diseases or disorders related to TF-mediated coagulation activity, thrombotic or coagulopathic related diseases or disorders or diseases or disorders such as inflammatory responses and chronic thromboembolic diseases or disorders associated with fibrin formation, including vascular disorders such as deep venous thrombosis, arterial thrombosis, post surgical thrombosis, coronary artery bypass graft (CABG), percutaneous transdermal coronary angioplasty (PTCA), stroke, cancer, tumour metastasis, angiogenesis, ischemia/reperfusion, arthritis including rheumatoid arthritis, thrombolysis, vascular restenosis, arteriosclerosis and restenosis following angioplasty, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis, and other diseases. The TF related diseases or disorders are not limited to in vivo coagulopatic disorders such as those named above, but includes ex vivo TF/FVIIa related processes such as coagulation that may result from the extracorporeal circulation of blood, including blood removed in-line from a patient in such processes as dialysis procedures, blood filtration, or blood bypass during surgery.

The TF dimer antagonist may be administered in pharmaceutically acceptable acid addition salt form or, where appropriate, as a alkali metal or alkaline earth metal or lower alkylammonium salt. Such salt forms are believed to exhibit approximately the same order of activity as the free base forms.

Apart from the pharmaceutical use of the compounds, they may be useful in vitro tools for investigating the inhibition of FVIIa, FXa or TF/FVIIa/FXa activity.

Pharmaceutical Compositions

In another aspect, the present invention includes within its scope pharmaceutical compositions comprising a TF dimer antagonist, as an active ingredient, or a pharmaceutically acceptable salt thereof together with a pharmaceutically acceptable carrier or diluent.

Optionally, the pharmaceutical composition of the invention may comprise a TF dimer antagonist in combination with one or more other compounds exhibiting anticoagulant activity, e.g., platelet aggregation inhibitor.

The compounds of the invention may be formulated into pharmaceutical composition comprising the compounds and a pharmaceutically acceptable carrier or diluent. Such carriers include water, physiological saline, ethanol, polyols, e.g., glycerol or propylene glycol, or vegetable oils. As used herein, “pharmaceutically acceptable carriers” also encompasses any and all solvents, dispersion media, coatings, antifungal agents, preservatives, isotonic agents and the like. Except insofar as any conventional medium is incompatible with the active ingredient and its intended use, its use in the compositions of the present invention is contemplated.

The compositions may be prepared by conventional techniques and appear in conventional forms, for example, capsules, tablets, solutions or suspensions. The pharmaceutical carrier employed may be a conventional solid or liquid carrier. Examples of solid carriers are lactose, terra alba, sucrose, talc, gelatine, agar, pectin, acacia, magnesium stearate and stearic acid. Examples of liquid carriers are syrup, peanut oil, olive oil and water. Similarly, the carrier or diluent may include any time delay material known to the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

The pharmaceutical compositions can be sterilised and mixed, if desired, with auxiliary agents, emulsifiers, salt for influencing osmotic pressure, buffers and/or colouring substances and the like, which do not deleteriously react with the active compounds.

The route of administration may be any route, which effectively transports the active compound to the appropriate or desired site of action, such as oral or parenteral, e.g., rectal, transdermal, subcutaneous, intranasal, intramuscular, topical, intravenous, intraurethral, ophthalmic solution or an ointment, the oral route being preferred.

If a solid carrier for oral administration is used, the preparation can be tabletted, placed in a hard gelatine capsule in powder or pellet form or it can be in the form of a troche or lozenge. The amount of solid carrier may vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

For nasal administration, the preparation may contain a compound of formula (I) dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet, which may be prepared by conventional tabletting techniques, contains

| | | |
|---|---|---|
| Core: | | |
| Active compound (as free compound | 10 | mg |
| or salt thereof) | | |
| Colloidal silicon dioxide (Areosil ®) | 1.5 | mg |
| Cellulose, microcryst. (Avicel ®) | 70 | mg |
| Modified cellulose gum (Ac-Di-Sol ®) | 7.5 | mg |
| Magnesium stearate | | |
| Coating: | | |
| HPMC | approx. 9 | mg |
| *Mywacett ® 9-40 T | approx. 0.9 | mg |

*Acylated monoglyceride used as plasticizer for film coating.

The compounds of the invention may be administered to a mammal, especially a human in need of such treatment, prevention, elimination, alleviation or amelioration of various thrombolytic or coagulophatic diseases or disorders as mentioned above. Such mammals also include animals, both domestic animals, e.g. household pets, and non-domestic animals such as wildlife.

Usually, dosage forms suitable for oral, nasal, pulmonal or transdermal administration comprise from about 0.001 mg to about 100 mg, preferably from about 0.01 mg to about 50 mg of the compounds of formula I admixed with a pharmaceutically acceptable carrier or diluent.

The compounds may be administered concurrently, simultaneously, or together with a pharmaceutically acceptable carrier or diluent, whether by oral, rectal, or parenteral (including subcutaneous) route. The compounds are often, and preferably, in the form of an alkali metal or earth alkali metal salt thereof.

Suitable dosage ranges varies as indicated above depending upon the exact mode of administration, form in which administered, the indication towards which the administration is directed, the subject involved and the body weight of the subject involved, and the preference and experience of the physician or veterinarian in charge.

The compounds of the present invention have interesting pharmacological properties. For example, the compounds of this invention can be used to modulate and normalise an impaired haemostatic balance in mammals caused by deficiency or malfunction of blood clotting factors or their inhibitors. The FVIIa and in particular the TF/FVIIa activity plays an important role in the control of the coagulation cascade, and modulators of this key regulatory activity such as the present invention can be used in the treatment of or prophylaxis of thrombotic or coagulopathic related diseases or disorders including vascular diseases and inflammatory responses. The pharmaceutical composition of the invention may thus be useful for modulating and normalising an impaired haemostatic balance in a mammal. In particular, the pharmaceutical composition may be useful for the treatment of or prophylaxis of thrombotic or coagulopathic related diseases or disorders including vascular diseases and inflammatory responses.

“Modulating and normalising an impaired haemostatic balance” means achieving an effect on the coagulation system measurable in vitro assays and/or animal models which diminishes the risk for thrombosis or bleedings.

More particularly, the pharmaceutical composition may be useful as an inhibitor of blood coagulation in a mammal, as an inhibitor of clotting activity in a mammal, as an inhibitor of deposition of fibrin in a mammal, as an inhibitor of platelet deposition in a mammal, in the treatment of mammals suffering from deep venous thrombosis, arterial thrombosis, post surgical thrombosis, coronary artery bypass graft (CABG), percutaneous transdermal coronary angioplasty (PTCA), stroke, tumour metastasis, inflammation, septic chock, hypotension, ARDS, pulmonary embolism, disseminated intravascular coagulation (DIC), vascular restenosis, platelet deposition, myocardial infarction, angiogenesis, or the prophylactic treatment of mammals with atherosclerotic vessels at risk for thrombosis. The compositions of the invention may also be used as an adjunct in thrombolytic therapy.

Furthermore the invention relates to a method for inhibiting the TF initiation activity in a mammal which method comprises administering an effective amount of at least one compound of the present invention, in combination with a pharmaceutical acceptable excipient and/or carrier to the mammal in need of such a treatment.

Assays

Inhibition of FVIIa/Phospholipids-Embedded TF-Catalyzed Activation of FX by Dimeric TF Antagonists FXa Generation Assay (Assay 1):

In the following example all concentrations are final. Lipidated TF (10 pM), FVIIa (100 pM) and dimeric TF antagonist or FFR-rFVIIa (0-50 nM) in HBS/BSA (50 mM hepes, pH 7.4, 150 mM NaCl, 5 mM CaCl₂, 1 mg/ml BSA) are incubated 60 min at room temperature before FX (50 nM) is added. The reaction is stopped after another 10 min by addition of ½ volume stopping buffer (50 mM Hepes, pH 7.4, 100 mM NaCl, 20 mM EDTA). The amount of FXa generated is determined by adding substrate S2765 (0.6 mM, Chromogenix, and measuring absorbance at 405 nm continuously for 10 min. IC₅₀ values for TF antagonist inhibition of FVIIa/lipidated TF-mediated activation of FX may be calculated. The IC50 value for FFR-rFVIIa is 51±26 pM in this assay.

Inhibition of FVIIa/cell Surface TF-Catalyzed Activation of FX by Dimeric TF Antagonists (Assay 2):

In the following example all concentrations are final. Monolayers of human lung fibroblasts WI-38 (ATTC No. CCL-75) or human bladder carcinoma cell line J82 (ATTC No. HTB-1) or human keratinocyte cell line CCD 1102KerTr (ATCC no. CRL-2310) constitutively expressing TF are employed as TF source in FVIIa/TF catalyzed activation of FX. Confluent cell monolayers in a 96-well plate are washed one time in buffer A (10 mM Hepes, pH 7.45, 150 mM NaCl, 4 mM KCl, and 11 mM glucose) and one time in buffer B (buffer A supplemented with with 1 mg/ml BSA and 5 mM Ca²⁺). FVIIa (1 nM), FX (135 nM) and varying concentrations of dimeric TF antagonist or FFR-rFVIIa in buffer B are simultaneously added to the cells. FXa formation is allowed for 15 min at 37° C. 50-μl aliquots are removed from each well and added to 50 μl stopping buffer (Buffer A supplemented with 10 mM EDTA and 1 mg/ml BSA). The amount of FXa generated is determined by transferring 50 μl of the above mixture to a microtiter plate well and adding 25 μl Chromozym X (final concentration 0.6 mM) to the wells. The absorbance at 405 nm is measured continuously and the initial rates of colour development are converted to FXa concentrations using a FXa standard curve. The IC50 value for FFR-rFVIIa is 1.5 nM in this assay.

Inhibition of ¹²⁵I-FVIIa Binding to Cell Surface TF by Dimeric TF Antagonists (Assay 3):

In the following example all concentrations are final. Binding studies are employed using the human bladder carcinoma cell line J82 (ATTC No. HTB-1) or the human keratinocyte cell line (CCD1102KerTr ATCC No CRL-2310) or NHEK P166 (Clonetics No. CC-2507) all constitutively expressing TF. Confluent monolayers in 24-well tissue culture plates are washed once with buffer A (see assay 8) supplemented with 5 mM EDTA and then once with buffer A and once with buffer B (see assay 8). The monolayers are preincubated 2 min with 100 μl cold buffer B. Varying concentrations of Mabs (or FFR-FVIIa) and radiolabelled FVIIa (0.5 nM ¹²⁵I-FVIIa) are simultaneously added to the cells (final volume 200 μl). The plates are incubated for 2 hours at 4° C. At the end of the incubation, the unbound material is removed, the cells are washed 4 times with ice-cold buffer B and lysed with 300 μl lysis buffer (200 mM NaOH, 1% SDS and 10 mM EDTA). Radioactivity is measured in a gamma counter (Cobra, Packard Instruments). The binding data are analyzed and curve fitted using GraFit4 (Erithacus Software, Ltd., (U.K.). The IC50 value for FFR-rFVIIa is 4 nM in this assay.

The present invention is further illustrated by the following examples.

The present invention is not to be limited in scope by the specific embodiments disclosed in the examples which are intended as illustrations of a number of aspects of the invention and any embodiments which are functionally equivalent are within the scope of this invention. Those skilled in the art will know, or be able to ascertain using no more than routine experimentation, many equivalents to the specific embodiments of the invention described herein. These and all other equivalents are intended to be encompassed by the following claims.

### EXAMPLES

The following LM comprising FVIIa inhibitors were synthesised:

| | |
|---|---|
| 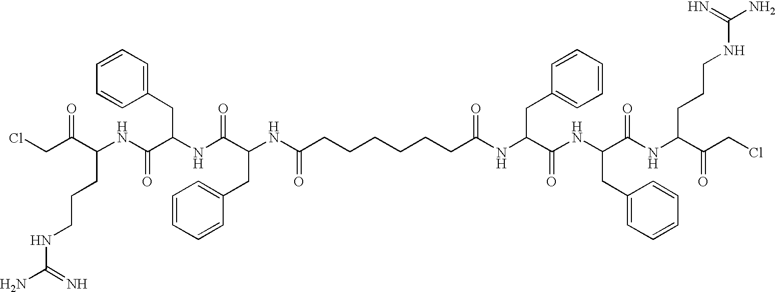 | Octanedioic acid bis-({1-[1-(1-chloro- acetyl-4-guanidino- butylcarbamoyl)-2- phenyl-ethylcar- bomoyl]-2-phenyl- ethyl-}-amide) (Example 1) |
| 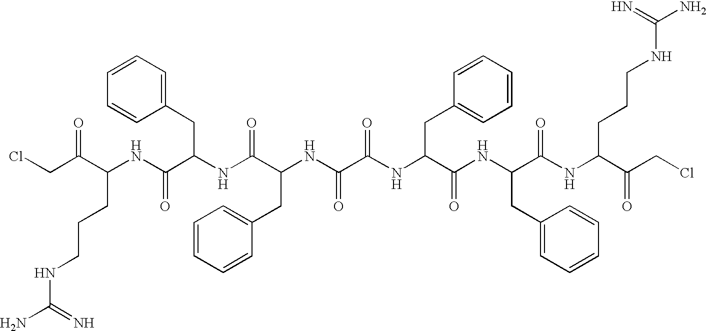 | N,N′-bis-(1-{1-[1- (2-chloro-acetyl)-4- guanidino-butylcar- bomoyl]-2-phenyl- ethylcarbomoyl}-2- phenylethyl)-oxal- amide-(Example 2) |
| 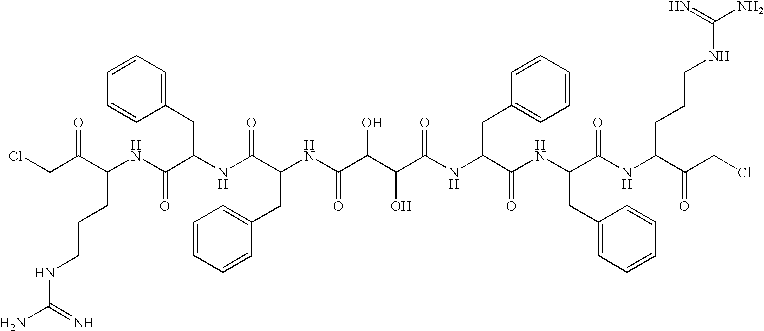 | N,N′-bis-(1-{1-[1- (2-chloro-acetyl)-4- guanidino-butylcar- bomoyl]-2-phenyl- ethylcarbomoyl}-2- phenylethyl-2,3- dihydroxysuccin- amide (Example 3) |
| 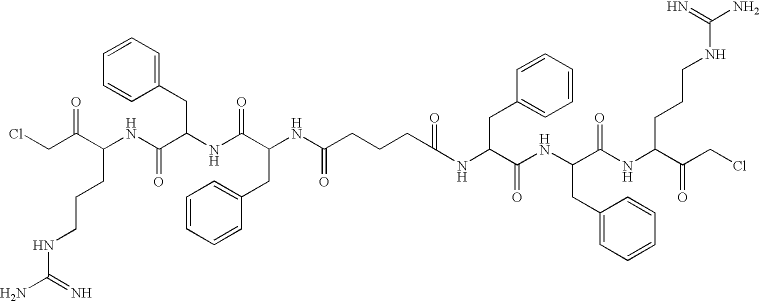 | Pentanedioic acid bis-[(1-{1-[1-(2- chloro-acetyl)-4- guanidino-butylcar- bamoyl]-2-phenyl- ethylcarbomoyl}-2- phenyl-ethyl)- amide](Example 4) |
| 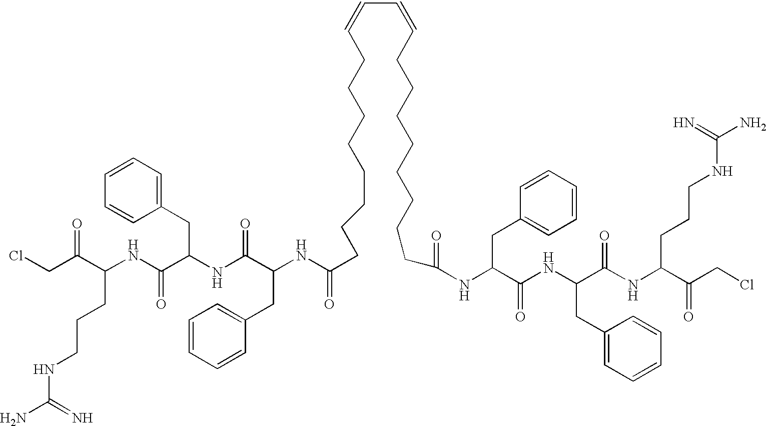 | Docosa-10,12-diy- nedioic acid bis-[(1- {1-(1-[1-(2-chloro- acetyl)-4-guanidino- butylcarbamoyl]-2- phenyl-ethylcar- bamoyl}-phenyl- ethyl)-amide](Example 5) |
| 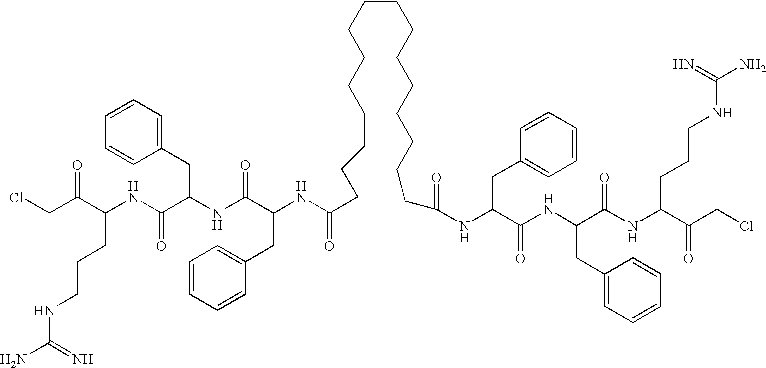 | Octanedioic acid bis-[(1-{1-[1-(2- chloro-acetyl)-4- guanidino-butylcar- bamoyl]-2-phenyl- ethylcarbomoyl}-2- phenyl-ethyl)- amide](Example 8) |
| 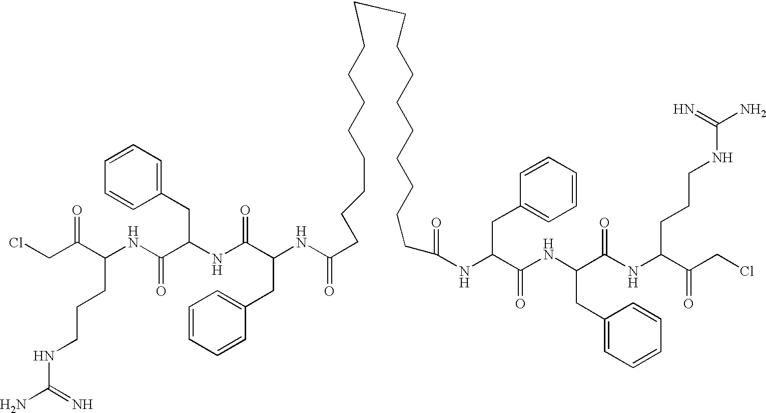 | Eicosanedioic acid bis-[(1-{1-[1-(2- chloro-acetyl)-4- guanidino-butylcar- bamoyl]-2-phenyl- ethylcarbamoyl}-2- 2-phenyl-ethyl)- amide](Example 7) |
| 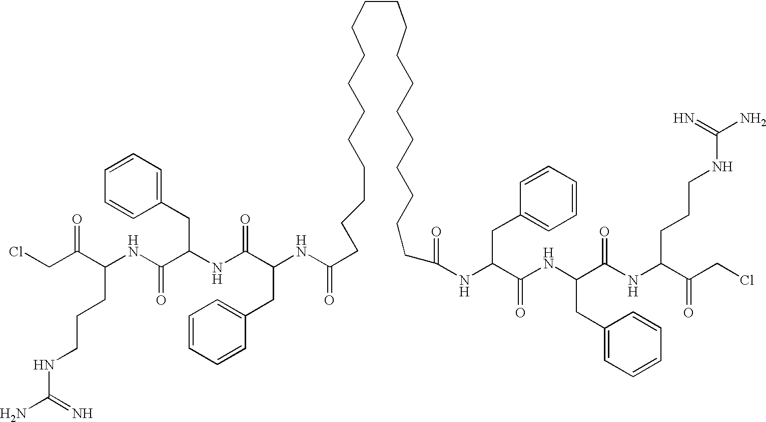 | Docosanedioic acid bis-[(1-{1-[1-(2- chloro-acetyl)-4- guanidino-butylcar- bamoyl]-2-phenyl- ethylcarbamoyl}-2- 2-phenyl-ethyl)- amide](Example 6) |

### Example 1

Octanedioic Acid bis-({1-[1-(1-chloroacetyl-4-guanidino-butylcarbamoyl)-2-phenyl-ethylcarbamoyl]-2-phenyl-ethyl}-amide) (1)

Disuccinimidyl suberate (Pierce #21555) (5.86 mg) was mixed with FFR-CMK (25 mg) in 1.5 ml of phosphate buffer pH 7.4, after addition of two drops of DMF the mixture was stirred in a closed vessel under N2 atmosphere for 1 day, subsequent evaporation yielded an yellow oil raw product (35 mg) which was purified on HPLC (reversed-phase column (Symmetry Shield,C₈, Waters, Part no. WAT200655)) with a constant flow of 1 ml/min. Elution was accomplished by increasing the percentage of organic phase (acetonitrile contaning 0.1% trifluoroacetic acid (TFA)) relative to aqueous phase (0.1% TFA in H₂O). A linear gradient from 14% to 50% organic phase over 35 min was used where the dimeric form of FFR-CMK was eluted at about 28 min.)

The fraction at rt (retention time) 30.94 min was isolated. MS (M+1) 1141 yield 12% oil.

### Example 2

N,N′-Bis-(1-{1-[1-(2-chloro-acetyl)-4-guanidino-butylcarbamoyl]-2-phenyl-ethylcarbamoyl}-2-phenylethyl)-oxalamide (2)

Disuccinimidyl oxalate (4.2 mg) and FFR-CMK (25 mg) were mixed in 1.5 ml of phosphate buffer pH 7.4, after addition of two drops of DMF the mixture was stirred in a closed vessel under N2 atmosphere for 2 days, subsequent evaporation yielded an yellow oil raw product which was purified on HPLC (reversed-phase column (SymmetryShild,C₈, Waters, Part no. WAT200655)) with a constant flow of 1 ml/min. Elution was accomplished by increasing the percentage of organic phase (acetonitrile contaning 0.1% trifluoroacetic acid (TFA)) relative to aqueous phase (0.1% TFA in H₂O). A linear gradient from 14% to 50% organic phase over 35 min was used where the dimeric form of FFR-CMK was eluted at about 28 min.)

The fraction at rt 31.08 min was isolated. MS (M+1) 1057, yield 8% yellow oil.

### Example 3

N,N′-Bis-(1-{1-[1-(2-chloro-acetyl)-4-guanidino-butylcarbamoyl]-2-phenyl-ethylcarbamoyl}-2-phenylethyl-2,3-dihydroxysuccinamide (3)

Disuccinimidyl tartrate (Pierce #20589) (5.16 mg) and FFR-CMK (25 mg) were mixed in 1.5 ml of phosphate buffer pH 7.4, after addition of 4 drops of DMF the mixture was stirred in a closed vessel under N2 atmosphere overnight, subsequent evaporation yielded an yellow oil raw product which was purified on HPLC (reversed-phase column (Symmetry-Shild,C₈, Waters, Part no. WAT200655)) with a constant flow of 1 ml/min. Elution was accomplished by increasing the percentage of organic phase (acetonitrile contaning 0.1% trifluoroacetic acid (TFA)) relative to aqueous phase (0.1% TFA in H₂O). A linear gradient from 14% to 50% organic phase over 35 min was used where the dimeric form of FFR-CMK was eluted at about 28 min.)

The fraction at rt 29.20 min was isolated. MS (M+1) 1117, yield 4% yellow oil.

### Example 4

Pentanedioic Acid bis-[(1-{1-[1-(2-chloro-acetyl)-4-guanidino-butylcarbamoyl]-2-phenyl-ethylcarbamoyl}-2-phenyl-ethyl)-amide] (4)

Disuccinimidyl glutarate (Pierce #20593) (4.89 mg) and FFR-CMK (25 mg) were mixed in 1.5 ml of phosphate buffer pH 7.4, after addition of 1 ml of DMF the mixture was stirred in a closed vessel under N2 atmosphere for 3 days, subsequent evaporation yielded an yellow oil raw product which was purified on HPLC (reversed-phase column (Symmetry-Shild,C₈, Waters, Part no. WAT200655)) with a constant flow of 1 ml/min. Elution was accomplished by increasing the percentage of organic phase (acetonitrile contaning 0.1% trifluoroacetic acid (TFA)) relative to aqueous phase (0.1% TFA in H₂O). A linear gradient from 14% to 50% organic phase over 35 min was used where the dimeric form of FFR-CMK was eluted at about 28 min.)

The fraction at rt 29.20 min was isolated. MS (M+1) 1099, yield 18% yellow oil.

### Example 5

10,12-docosadiyndioic acid bis-({1-[1-(1-chloroacetyl-4-guanidino-butylcarbamoyl)-2-phenyl-ethylcarbamoyl]-2-phenyl-ethyl}-amide)

Docosa-10,12-diynedioic acid bis-(2,5-dioxo-pyrrolidin-1-yl)ester (9.4 mg) is mixed with FFR-CMK (25 mg) in 1.0 mL of phosphate buffer pH 7.4, after addition of 1 mL of DMF the mixture was stirred in a closed vessel under N2 atmosphere for 4 days Subsequent evaporation yielded an yellow oil raw product.

### Example 6

Docosanedioic acid bis-({1-[1-(1-chloroacetyl-4-guanidino-butylcarbamoyl)-2-phenyl-ethylcarbamoyl]-2-phenyl-ethyl}-amide)

Docosanedioic acid bis-(2,5-dioxo-pyrrolidin-1-yl)ester (9.5 mg) is mixed with FFR-CMK (25 mg) in 1.o mL of phosphate buffer pH 7.4, after addition of 1 mL of DMF the mixture was stirred in a closed vessel under N2 atmosphere for 4 days. Subsequent evaporation yielded an yellow oil raw product

### Example 7

Icosanedioic acid bis-({1-[1-(1-chloroacetyl-4-guanidino-butylcarbamoyl)-2-phenyl-ethylcarbamoyl]-2-phenyl-ethyl}-amide)

Eicosanedioic acid bis-(2,5-dioxo-pyrrolidin-1-yl)ester (9.1 mg) is mixed with FFR-CMK (25 mg) in 1.0 mL of phosphate buffer pH 7.4, and 1 mL of DMF the mixture was subsequently stirred in a closed vessel under N2 atmosphere for 4 days subsequent evaporation yielded an yellow oil raw product.

### Example 8

Octadecadioic acid bis-({1-[1-(1-chloroacetyl-4-quanidino-butylcarbamoyl)-2-phenyl-ethylcarbamoyl]-2-phenyl-ethyl}-amide)

Octadecanedioic acid bis-(2,5-dioxo-pyrrolidin-1-yl)ester (8.6 mg) is mixed with FFR-CMK (25 mg) in ) in 1.0 mL of phosphate buffer pH 7.4, and 1 mL of DMF the mixture was subsequently stirred in a closed vessel under N2 atmosphere for 4 days. Evaporation yielded an yellow oil raw product.

### Example 9

Octanedioic acid bis-({1-[1-(1-chloroacetyl-4-guanidino-butylcarbamoyl)-2-phenyl-ethylcarbamoyl]-2-phenyl-ethyl}-amide was mixed with rFVIIa in 10 mM glycyl-glycine pH 7.5 containing 150 mM NaCl and 10 mM CaCl₂ at a molar ration of 1:2, and incubated 6 days at 4° C. The sample was loaded on a HiLoad 16/60 Superdex 75 column (Pharmacia) equilibrated with 20 mM TrisCl, 250 mM NaCl, 5 mM CaCl₂, pH 8.0. The fractions were analyzed by SDS-PAGE (Novex), and fraction number 16-19 showing a band of molecular mass 100,000 were pooled and concentrated on a Centiprep 10 column (Amicon). The concentrated sample was loaded on a HiLoad 16/60 Superdex 200 column (Pharmacia) equilibrated with 20 mM TrisCl, 250 mM NaCl, 5 mM CaCl₂, pH 8.0, and fractions containing a band of molecular mass 100,000 on SDS-PAGE were pooled and concentrated.

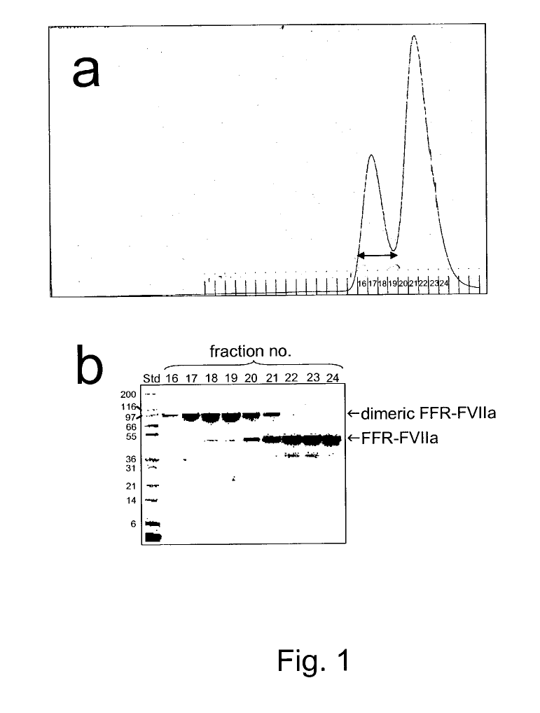

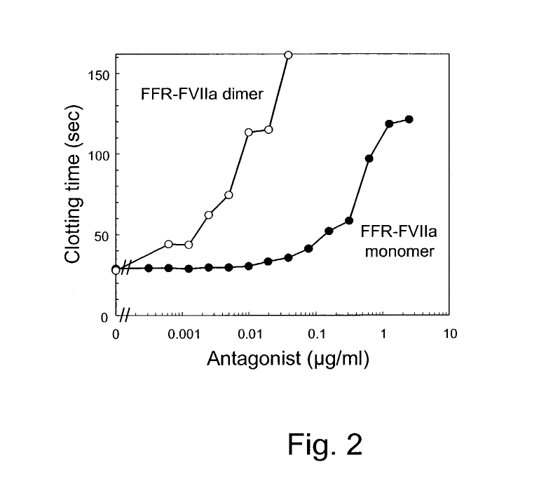

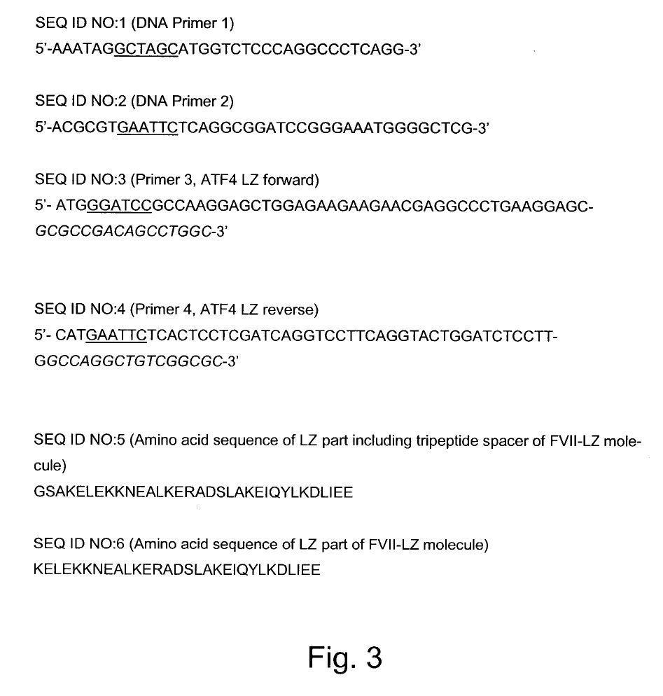

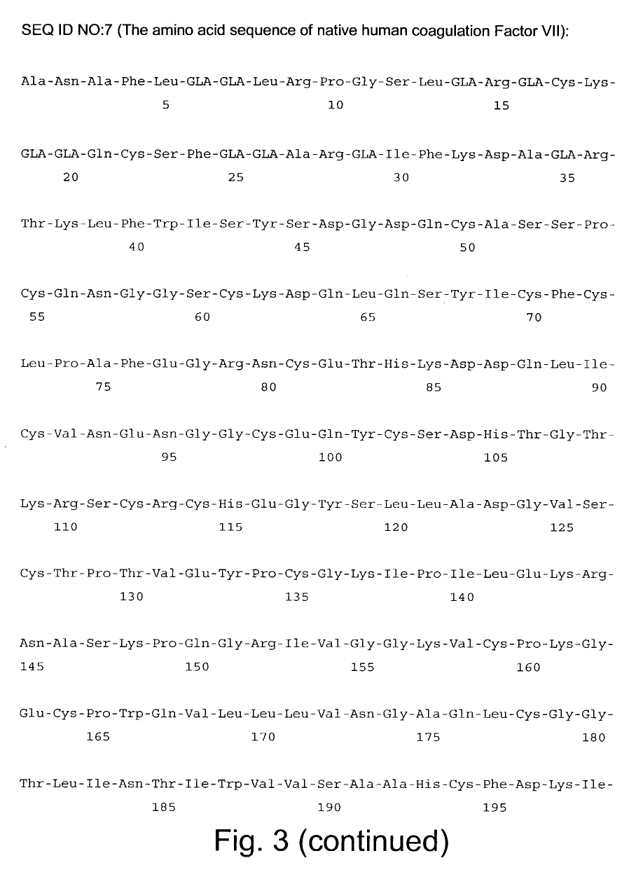

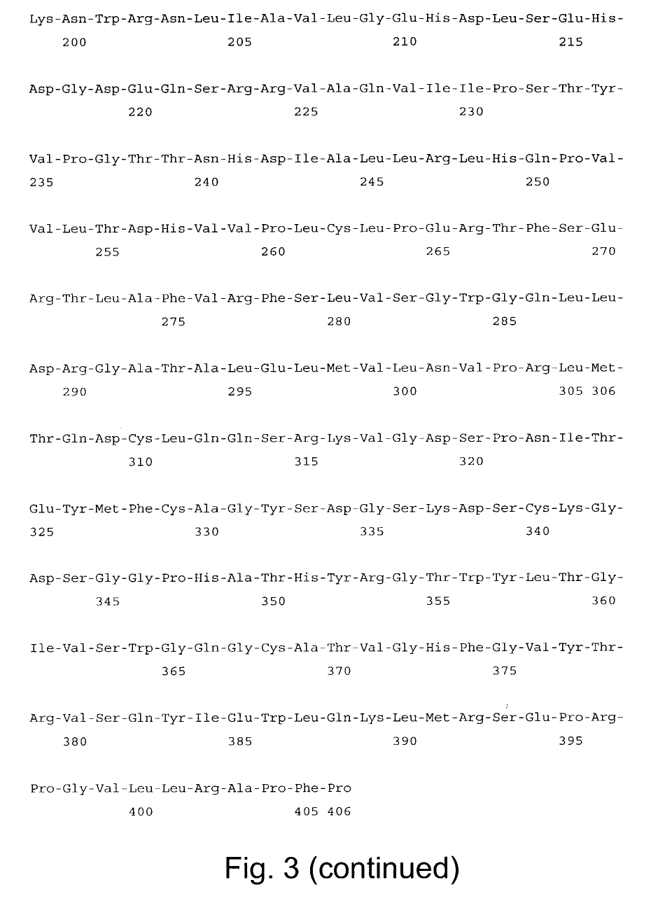

Size exclusion chromatography of rFVIIa reacted with octanedioic acid bis-({1-[1-(1-chloroacetyl-4-guanidino-butylcarbamoyl)-2-phenyl-ethylcarbamoyl]-2-phenyl-ethyl}-amide (FIG. 1a), and SDS-PAGE of the fractions (FIG. 1b). Samples for SDS-PAGE were unreduced. MARK 12 (Invitrogen) was used as molecular mass standard (left lane). Fraction no 16-19 were pooled, concentrated, and loaded on a Superdex 200 column.

### Example 10

Generation and expression of FVII-LZ.

A plasmid vector pFVII-fus for expression of human FVII fusion proteins in mammalian cells is generated based on the pCI-neo plasmid vector (Promega, Madison, Wis.) in which the existing BamHI site has been deleted by BamHI digest followed by T4 DNA polymerase fill-in to yield the plasmid vector termed pCI-neo (ΔBamHI). Briefly, the pFVII-fus vector carries the cDNA nucleotide sequence encoding human FVII including the propeptide under the control of a strong CMV promoter for transcription of the inserted cDNA, and neomycin phosphotransferase cDNA under the control of an SV40 early promoter for use as a selectable marker. The FVII insert is generated from the I.M.A.G.E. clone ID: 1848877, but any full-length FVII cDNA can be used, by PCR using Expand High Fidelity (Roche). The PCR product is generated using the oligonucleotides Primer 1 and 2, by procedures for preparing a DNA construct using polymerase chain reaction using specific primers are well known to persons skilled in the art (cf. PCR Protocols, 1990, Academic Press, San Diego, Calif., USA):

Primer 1. 5′-AAATAGGCTAGCATGGTCTCCCAGGCCCTCAGG-3′ (SEQ ID NO: 1)

Primer 2. 5′-ACGCGTGMTTCTCAGGCGGATCCGGGAAATGGGGCTCG-3′ (SEQ ID NO: 2)

The resulting product contains the entire FVII coding sequence followed by an inframe segment encoding Gly-Ser-Ala which contains a BamHI site used for in frame insertion of C-terminal tags. The fragment is digested by NheI and EcoRI (underlined in the primer sequence) and introduced into the NheI and EcoRI sites of pCI-neo (ΔBamHI) to yield the plasmid vector termed pFVII-fus.

Different leucine zipper inserts are generated as a primer dimer using overlapping oligonucleotides (overlapping region in italics) which are filled-in using T4 or Taq DNA polymerase to yield a double-stranded DNA fragment encoding the LZ of choice preceded by a BamHI site encoding Gly-Ser-Ala and followed by a stop codon and an EcoRI site (underlined in the primer sequences). Thus, an example of a design resulting in a fusion protein containing an ATF4 derived LZ would look as follows.

| | |
|---|---|
| Primer 3 (ATF4 LZ forward): | |
| 5′-ATGGGATCCGCCAAGGAGCTGGAGAAGAAGAACGAGGCCCTGAAGGAGCGCGCCGACAGCCTGGC-3′ | (SEQ ID NO:3) |
| Primer 4 (ATF4 LZ reverse): | |
| 5′-CATGAATTCTCACTCCTCGATCAGGTCCTTCAGGTACTGGATCTCCTTGGCCAGGCTGTCGGCGC-3′ | (SEQ ID NO:4) |

Following the fill-in reaction with T4 polymerase, the DNA polymerase is removed and the dsDNA product is digested with BamHI and EcoRI and the resulting insert is purified using conventional methods. The purified insert is ligated into the BamHI and EcoRI site of pFVII-fus vector using T4 DNA ligase and transformed into an appropriate E. coli strain, e.g. DH5α or XL1Blue; plasmid vectors containing the desired LZ tag (termed pFVII-LZ) are identified and isolated using standard techniques and the sequence is verified DNA sequencing. The resulting expression vector will encode the following protein:

FVII-GSA-KELEKKNEALKERADSLAKEIQYLKDLIEE

The pFVII-LZ vector is transfected into CHO cells using Lipofectamine, or similar technique, and stable clones are isolated following neomycin selection. Clones secreting FVII is identified using a FVII ELISA and any high producing clones will be further subcloned to yield a clone with a high specific FVII expression in Dulbecco-modified Eagle's medium with 10% fetal calf serum. The clone will subsequently be adapted to serum free suspension culture using a commercially available CHO medium (JRH Bioscience). The resulting recombinant FVII-LZ material can then be purified from the media using conventional methods (Thim, L. et al. Biochemistry (1988) 27:7785-93).

### Example 11

Expression of FVII-LZ.

BHK cells are transfected with the pFVII-LZ vector essentially as previously described (Thim et al. (1988) Biochemistry 27, 7785-7793; Persson and Nielsen (1996) FEBS Lett. 385, 241-243) to obtain expression of FVII-LZ. The FVII-LZ protein is purified as follows:

Conditioned medium is loaded onto a 25-ml column of Q Sepharose Fast Flow (Pharmacia Biotech) after addition of 5 mM EDTA, 0.1% Triton X-100 and 10 mM Tris, adjustment of pH to 8.0 and adjustment of the conductivity to 10-11 mS/cm by adding water.

Elution of the protein is accomplished by stepping from 10 mM Tris, 50 mM NaCl, 0.1% Triton X-100, pH 8.0 to 10 mM Tris, 50 mM NaCl, 25 mM CaCl₂, 0.1% Triton X-100, pH 8.0. The fractions containing S314E/K316H-FVII are pooled and applied to a 25-ml column containing monoclonal antibody F1A2 (Novo Nordisk, Bagsvaerd, Denmark) coupled to CNBr-activated Sepharose 4B (Pharmacia Biotech).

The column is equilibrated with 50 mM Hepes, pH 7.5, containing 10 mM CaCl₂, 100 mM NaCl and 0.02% Triton X-100. After washing with equilibration buffer and equilibration buffer containing 2 M NaCl, bound material is eluted with equilibration buffer containing 10 mM EDTA instead of CaCl₂. Before use or storage, excess CaCl₂ over EDTA is added or FVII-LZ is transferred to a Ca²⁺-containing buffer. The yield of each step was followed by factor VII ELISA measurements and the purified protein was analysed by SDS-PAGE. Subsequent inactivation of the FVII moiety of FVII-LZ molecules by e.g. FFR-CMK are known to the person skilled in the art.

### Example 12

FFR-FVIIa dimer and FFR.FVIIa was analyzed in a competition clotting assay as follows: The FFR-FVIIa dimer prepared using Octanedioic acid bis-({1-[1-(1-chloroacetyl-4-guanidino-butylcarbamoyl)-2-phenyl-ethylcarbamoyl]-2-phenyl-ethyl}-amide as linker or FFR-FVIIa monomer was incubated 60 min at 37° C. at the indicated concentrations with 20 nM FVIIa, 10 nM CaCl₂, and lipidated recombinant TF (Innovin, Dade Behring) diluted 200× in 50 mM imidazole, 0.1 M NaCl, pH 7.4, with 0.1% BSA to give a clotting time of 30 sec in samples without antagonist. Clotting times were measured on an ACL 300 Research instrument (Instrumentation Laboratories) after adding 0.5 volume FVII-deficient plasma (Helena BioSciences). The concentration of FFR-FVIIa monomer required for doubling the clotting time was 0.23 μg/ml, while the concentration of FFR-FVIIa dimer required for doubling the clotting time was 0.002 μg/ml, i.e. the dimer was appr. 100× more efficient in prolonging the clotting time.

1. A compound having the formula A-(LM)-D, wherein A and D are FVII polypeptides that bind to tissue factor (TF); and LM is a linker moiety with a molecular weight less than 30,000 daltons; and wherein said compound inhibits TF activity, with the proviso that the compound does not have the formula: wtFVIIai-(DPTA-dim-FFR-cmk)-wtFVIIai or FVIIai(Q10E32)-(DPTA-dim-FFR-cmk)-FVIIai(Q10E32).

2. The compound according to claim 1, wherein the affinity of binding of said compound to TF is higher than the affinity of binding to TF of either A or D alone.

3. The compound according to claim 1, wherein A and D comprise the amino acid sequence of human recombinant FVIIa.

4. The compound according to claim 1, wherein A and D are human recombinant FVIIai.

5. The compound according to claim 1, wherein LM comprises an amino acid sequence.

6. The compound according to claim 5, wherein LM comprises a leucine zipper.

7. The compound according to claim 5, wherein LM comprises an amino acid sequence independently selected from the group consisting of SEQ ID NO: 5 and SEQ ID NO: 6.

8. The compound according to claim 5, wherein LM comprises an amino acid sequence (Gly-Gly-Gly-Gly-Ser)n, wherein n is any integer from 1 to 10.

9. The compound according to claim 1, wherein LM comprises a molecule selected from the group consisting of straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈cycloalkyl, a 3 to 8-membered cyclic ring comprising carbon and at least one N, O or S atom in the ring, aryl, heteroaryl, amino acid, wherein said molecule is optionally substituted with one or more of the following groups: H, hydroxy, phenyl, phenoxy, benzyl, thienyl, oxo, amino, C₁₄-alkyl, —CONH₂, —CSNH₂, C₁₋₄ monoalkylamino, C₁₋₄ dialkylamino, acylamino, sulfonyl, carboxy, carboxamido, halogeno, C₁₋₆ alkoxy, C₁₋₆ alkylthio, trifluoroalkoxy, alkoxycarbonyl, haloalkyl.

10. The compound according to claim 1, wherein LM comprises a divalent chloromethyl ketone inhibitor consisting of two monomers independently selected from the group comprising Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloro-methylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone.

11. The compound according to claim 1, wherein LM comprises two FVIIa inhibitors.

12. The compound according to claim 1, wherein LM is selected from the group consisting of octadecadioic acid bis-({1-[1-(1-chloroacetyl-4-guanidino-butylcarbamoyl)-2-phenyl-ethylcarbamoyl]-2-phenyl-ethyl}-amide), icosanedioic acid bis-({1-[1-(1-chloroacetyl-4-guanidino-butylcarbamoyl)-2-phenyl-ethylcarbamoyl]-2-phenyl-ethyl}-amide), docosanedioic acid bis-({1-[1-(1-chloroacetyl-4-guanidino-butylcarbamoyl)-2-phenyl-ethylcarbamoyl]-2-phenyl-ethyl}-amide), 10,12-docosadiyndioic acid bis-({1-[1-(1-chloroacetyl-4-guanidino-butylcarbamoyl)-2-phenyl-ethylcarbamoyl]-2-phenyl-ethyl}-amide) and octanedioic acid bis-({1-[1-(1-chloroacetyl-4-guanidino-butylcarbamoyl)-2-phenyl-ethylcarbamoyl]-2-phenyl-ethyl}-amide).

13. A method for reducing TF activity, said method comprising contacting a TF expressing cell with a compound having the formula A-(LM)-D, wherein A and D are FVII polypeptides that binds to TF; and LM is a linker moiety with a molecular weight less than 30,000 daltons; and wherein said compound inhibits TF activity, with the proviso that the compound does not have the formula wtFVIIai-(DPTA-dim-FFR-cmk)-wtFVIIai or FVIIai(Q10E32)-(DPTA-dim-FFR-cmk)-FVIIai(Q10E32).

14. A method according to claim 13, wherein A and D are human recombinant FVIIai.

15. A pharmaceutical composition comprising a compound having the formula A-(LM)-D, wherein A and D are FVII polypeptides that binds to TF; and LM is a linker moiety with a molecular weight less than 30,000 daltons; and wherein said compound inhibits TF activity, with the proviso that the compound does not have the formula wtFVIIai-(DPTA-dim-FFR-cmk)-wtFVIIai or FVIIai(Q10E32)-(DPTA-dim-FFR-cmk)-FVIIai(Q10E32); and a pharmaceutically acceptable carrier or excipient.

16. The pharmaceutical composition according to claim 15, wherein A and D are human recombinant FVIIai.

17. The pharmaceutical composition according to claim 15, further comprising a platelet aggregation inhibitor.

18. A compound for use as a medicament having the formula A-(LM)-D, wherein A and D are FVII polypeptides that binds to TF; and LM is a linker moiety with a molecular weight less than 30,000 daltons; and wherein said compound inhibits TF activity, with the proviso that the compound does not have the formula wtFVIIai-(DPTA-dim-FFR-cmk)-wtFVIIai or FVIIai(Q10E32)-(DPTA-dim-FFR-cmk)-FVIIai(Q10E32).

19. The compound according to claim 18, wherein A and D are human recombinant FVIIai.

20. Use of a compound having the formula A-(LM)-D, wherein A and D are FVII polypeptides that binds to TF; and LM is a linker moiety with a molecular weight less than 30,000 daltons; and wherein said compound inhibits TF activity with the proviso that the compound is not a compound having the formula of wtFVIIai-(DPTA-dim-FFR-cmk)-wtFVIIai or FVIIai(Q10E32)-(DPTA-dim-FFR-cmk)-FVIIai(Q10E32) for the manufacture of a medicament for preventing or treating a TF related diseases or disorders.

21. The use according to claim 20, wherein the compound having the formula A-(LM)-D is according to any one of the claims 1-12.

22. Use according to any one of claims 20-21, wherein the TF related diseases or disorders are deep venous thrombosis, arterial thrombosis, post surgical thrombosis, coronary artery bypass graft (CABG), percutaneous transdermal coronary angioplasty (PTCA), stroke, cancer, tumour metastasis, angiogenesis, ischemia/reperfusion, arthritis including rheumatoid arthritis, thrombolysis, arteriosclerosis and restenosis following angioplasty, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis.

23. A method for prevention or treatment of TF related diseases or disorders in a mammal, which method comprises administering to a mammal an effective amount for said prevention or treatment of at least one compound having the formula A-(LM)-D, wherein A and D are FVII polypeptides that bind to TF; and LM is a linker moiety with a molecular weight less than 30,000 daltons; and wherein said compound inhibits TF activity, with the proviso that the compound does not have the formula wtFVIIai-(DPTA-dim-FFR-cmk)-wtFVIIai or FVIIai(Q10E32)-(DPTA-dim-FFR-cmk)-FVIIai(Q10E32).

24. The method according to claim 23, wherein A and D are human recombinant FVIIai.

25. A method according to claim 23, wherein the TF related diseases or disorders are deep venous thrombosis, arterial thrombosis, post surgical thrombosis, coronary artery bypass graft (CABG), percutaneous transdermal coronary angioplasty (PTCA), stroke, cancer, tumour metastasis, angiogenesis, ischemia/reperfusion, arthritis including rheumatoid arthritis, thrombolysis, arteriosclerosis and restenosis following angioplasty, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis.


SEQUENCE LISTING (7 sequences)

SEQ ID NO: 1 - DNA, 33 bp, Artificial
  DNA Primer 1
aaataggctagcatggtctcccaggccctcagg

SEQ ID NO: 2 - DNA, 39 bp, Artificial
  DNA Primer 2
acgcgtgaattctcaggcggatccgggaaatggggctcg

SEQ ID NO: 3 - DNA, 65 bp, Artificial
  Primer 3, ATF4 LZ forward
atgggatccgccaaggagctggagaagaagaacgaggccctgaaggagcgcgccgacagcctggc

SEQ ID NO: 4 - DNA, 65 bp, Artificial
  Primer 4, ATF4 LZ reverse
catgaattctcactcctcgatcaggtccttcaggtactggatctccttggccaggctgtcggcgc

SEQ ID NO: 5 - protein, 33 aa, Artificial
  Amno acid sequence of LZ part including tripeptide spacer of FVII-LZ molecule
GSAKELEKKNEALKERADSLAKEIQYLKDLIEE

SEQ ID NO: 6 - protein, 30 aa, Artificial
  Amino acid sequence of LZ part of FVII-LZ molecule
KELEKKNEALKERADSLAKEIQYLKDLIEE

SEQ ID NO: 7 - protein, 406 aa, human coagulation Factor VII
  MISC_FEATURE (1)..(406): Xaa means 4-carboxyglutamic acid (gamma-carboxyglutamate)
ANAFLXXLRPGSLXRXCKXXQCSFXXARXIFKDAXRTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP